(19) United States
(12) Patent Application Publication
(10) Pub. No.: US 2004/0121407 A1
(43) Pub. Date: Jun. 24, 2004

(54) Regulation of the growth hormone/IGF-1 axis

(75) Inventors: Peter Distefano, Southboro, MA (US); Cynthia A. Bayley, Norwell, MA (US); L. Edward Cannon, Cambridge, MA (US)

Correspondence Address: FISH & RICHARDSON PC, 225 FRANKLIN ST, BOSTON, MA, 02110, US

(73) Assignee: ELIXIR PHARMACEUTICALS, INC.

(21) Appl. No.: 10/656,530

(22) Filed: Sep. 5, 2003

Related U.S. Application Data
(60) Provisional application No. 60/487,308, filed on Jul. 14, 2003.
(60) Provisional application No. 60/487,344, filed on Jul. 14, 2003.
(60) Provisional application No. 60/408,560, filed on Sep. 6, 2002.

(51) Int. Cl. G01N 33/00; G01N 33/53; G01N 33/543

(52) U.S. Cl. 435/7.1; 436/518; 800/3

(57) ABSTRACT

This disclosure provides, inter alia, treatments and compositions that alter life span regulation and cellular responses to diseases and disorders by antagonizing the GH/IGF-1 axis. Also provided are methods of screening for agents that can modulate the GH/IGF-1 axis.

### CROSS-REFERENCE TO RELATED APPLICATIONS

This application claims priority to U.S. Provisional Applications Serial Nos. 60/408,560, filed Sep. 6, 2002, 60/487,344, filed Jul. 14, 2003, and 60/487,308, filed Jul. 14, 2003, the contents of which are hereby incorporated by reference, for all purposes, in their entireties.

### BACKGROUND

Growth hormone (GH) is a 22 kDa, 191 amino acid single chain peptide containing two disulfide bridges. In humans, GH is essential for linear growth of the infant, child, and adolescent and also plays an important role in the regulation of metabolism. In mammals, it is the primary hormone responsible for growth, and it accelerates metabolic processes such as lipolysis and protein synthesis. GH and many other hormones are part of a complex endocrine system, called the GH/insulin-like growth factor-1 axis (GH/IGF-1 axis).

GH secretion and circulating IGF-1 levels are regulated by the GH/IGF-1 axis. Included in the GH/IGF-1 axis are hormones from the hypothalamus and from elsewhere in the body, receptors on the anterior pituitary and peripheral tissues and organs, anterior pituitary somatotrophs that produce and secrete GH, and peripheral tissues that secrete IGF-1 in response to GH. FIG. 1 is a schematic of the GH/IGF-1 axis.

### SUMMARY

In one aspect, the invention features a method of reducing the activity of the GH/IGF-1 axis in a subject, e.g., an adult subject (e.g., an adult mammal, e.g., an adult human or an adult non-human primate). The method includes administering a GH/IGF-1 axis modulator (e.g., an antagonist, an agonist, a partial agonist, or an inverse agonist of the axis) to a subject (e.g., an adult subject) at a dosage effective to cause a reduction in levels of GH, IGF-1 and/or IGF-1 receptor signaling in the subject by at least 20% (e.g., 30, 40, 50, 55, 60, or 80%) of a normal level for the chronological age of the adult subject, but not below detection. The reduction can include reducing a level to a resulting level that is less than 90, 80, 70, 60, 50, or 30% and/or greater 70, 65, 60, 55, 50, 45, 40, or 15% of the initial level of the subject. In another example, partial reduction can include reducing a level to a resulting level that is less than 90, 80, 70, 60, 50, or 30% and/or greater than 70, 65, 60, 55, 50, 45, 40, or 15% of the average level among normal individuals having the same age and gender as the subject.

In general, prior to the administering, the subject initially has normal levels of GH and/or IGF-1 for the chronological age of the subject. Typically, the subject is an adult (e.g., a human adult having an age of at least 18, 21, 24, or 28 years) without defects in the GH/IGF-1 axis, and thus does not have acromegaly. In an embodiment, the subject does not have diabetic retinopathy. In some embodiments, the adult subject has a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardio-vascular disorder.

In one embodiment, the modulator can be a compound, e.g., a small organic molecule (e.g., less than 7 kDa in molecular weight, e.g., 6, 5, 4, 3, 2, 1, 0.7, 0.6, 0.5, 0.4, 0.3, or 0.2 kDa) or macromolecule. In one embodiment, the modulator is a peptide, polypeptide, protein, antibody, antibody fragment, peptidomimetic, peptoid, nucleic acid, or other chemical compound or a combination of thereof. In one embodiment, the modulator is a compound that directly antagonizes a positively acting GH/IGF-1 axis component. In another embodiment, the modulator is a compound that directly agonizes an inhibitory GH/IGF-1 axis component.

For example, the compound can bind to and antagonize the GH receptor (e.g., Pegvisomant), bind to and antagonize the GHRH receptor, function as a somatostatin agonist (e.g., a somatostatin agonist, e.g., L-054,522, octreotide, lanreotide, vapreotide, and other analogues thereof), function as a GH secretagogue antagonist, or affect the GH/IGF-1 axis in any way such that the compound, when given at its appropriate dosage reduces GH, IGF-1 levels and/or IGF-1 receptor signaling in the subject by at least 30% (e.g., 30, 35, 40, 45, 50, 55, 60, 65, 70, 75, or 80%) of a normal level for the chronological age of the adult subject, but not below detection. In another embodiment, the compound is administered at regular intervals (e.g., daily, weekly, biweekly, or monthly). In yet another embodiment, the compound is administered at regular intervals for at least two months (e.g., at least six or nine months or for at least one, two, five, ten, 20, 25, or 30 years). The method can include other features described herein.

In another aspect, the invention provides a method of modulating lifespan regulation in a subject. The method includes:

a) evaluating and monitoring one or more indicators of GH/IGF-1 axis activity in an adult subject that does not have acromegaly or supranormal levels of GH or IGF-1 with respect to their chronological age; and

b) administering, to the subject, a regimen of doses of a compound that alters (e.g., inhibits or enhances) activity of a GH/IGF-1 axis component, the regimen being a function of the indicator or indicators and effective to maintain detectable, subnormal levels of IGF-1 in the subject, (e.g., a healthy subject) with respect to a subject of their age. The regimen of treatment administration can be adjusted as a function of the indicator or indicators as evaluated during the regimen and the evaluation of the indicator or indicators can be repeated more than once during the treatment regimen.

In one embodiment, the regimen causes a reduction in axis activity that reduces a level of GH, IGF-1, or IGF-1 receptor signaling to less than 90, 80, 70, 60, 50, 40 or 30%. For example, the regimen may reduce activity, but not below the level of detection, e.g., not below 15, 20, 25, 30, 35, or 40% of the initial level (prior to the regimen) of the subject. In another example, the reduction can include reducing such levels to a resulting level that is less than 90, 80, 70, 60, 50, 40, or 30% and/or greater than 70, 65, 60, 55, 50, 45, 40, or 15% of the average level among normal individuals having the same age and gender as the subject.

Prior to the administering, the adult typically has normal levels of GH and/or IGF-1 for the chronological age of the adult subject. Typically, the subject is an adult (e.g., a human adult having an age of at least 18, 21, 24, or 28 years) without defects in the GH/IGF-1 axis, and thus does not have acromegaly. In one embodiment, the subject does not have diabetic retinopathy. In some embodiments, the adult subject has a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardio-vascular disorder.

The compound can be a small organic molecule (e.g., less than 7 kDa in molecular weight, e.g., 6, 5, 4, 3, 2, 1, 0.7, 0.6, 0.5, 0.4, 0.3, or 0.2 kDa). The compound can also be a peptide, polypeptide, antibody, antibody fragment, peptidomimetic, peptoid, nucleic acid, or other chemical compound or a combination of any of these. For example, the compound can bind to and antagonize the GH receptor (e.g., Pegvisomant), bind to and antagonize the GHRH receptor, function as a somatostatin agonist (e.g., a somatostatin agonist, e.g., L-054,522, octreotide, lanreotide, vapreotide, and other analogues thereof), function as a GH secretagogue antagonist, or affect the GH/IGF-1 axis in any way such that the compound, when given at its appropriate dosage reduces GH, IGF-1 levels and/or IGF-1 receptor signaling in the subject by at least 30% (e.g., 30, 35, 40, 45, 50, 55, 60, 65, 70, 75, or 80%) of a normal level for the chronological age of the adult subject, but not below detection. An exemplary antagonist of GHS or the GHS-R is a modified peptide, e.g., [D-Lys³]-GHRP-6, which can also be used to modulate the axis.

In another embodiment, the compound is administered at regular intervals (e.g., daily, weekly, biweekly, or monthly). In yet another embodiment, the compound is administered at regular intervals for at least two months (e.g., at least six or nine months or for at least one, two, five, ten, 20, 25, or 30 years). The method can include other features described herein.

In another aspect, the invention features a method for treating or preventing an age-associated disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for the age-associated disorder. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of the age-associated disorder, and/or providing a treatment regimen to the subject for a period of greater than one, two, four, six, or twelve months. The method can also include other features described herein.

In another aspect, the invention features a method for treating or preventing a geriatric disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a geriatric disorder. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of the geriatric disorder, and/or providing a treatment regimen to the subject for a period of greater than one, two, four, six, or twelve months. The method can also include other features described herein.

In another aspect, the invention features a method for treating or preventing a disorder having an age-associated susceptibility factor. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a disorder having an age-associated susceptibility factor. Exemplary disorders having an age-associated susceptibility factor include bacterial and viral disorders (e.g., pneumonia) and certain injuries, e.g., bone injuries, e.g., fracture, fracture morbidity, frailty and co-morbidity associated with such injuries, which disorders have increased severity or mortality among adults greater than 80 years of age. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of the disorder having an age-associated susceptibility factor, and/or providing a treatment regimen to the subject for a period of greater than one, two, four, six, or twelve months. The method can also include other features described herein.

In another aspect, the invention features a method for treating or preventing a neoplastic disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for the neoplastic disorder. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of the neoplastic disorder, and/or providing a treatment regimen to the subject for a period of greater than one, two, four, six, or twelve months. Exemplary neoplastic disorders include hypertrophies and cancers, such as solid tumors, soft tissue tumors, hematopoietic cancers, and metastatic cancers. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. The method can also include other features described herein.

In another aspect, the invention features a method for treating or preventing a non-neoplastic disorder, e.g., an age-associated non-neoplastic disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a non-neoplastic disorder. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a non-neoplastic disorder, and/or providing a treatment regimen to the subject for a period of greater than one, two, four, six, or twelve months. The method can also include other features described herein.

In another aspect, the invention features a method for treating or preventing a neurological disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a neurological disorder. Exemplary neurological disorders include neuropathies, muscular atrophy, and neurodegenerative disorders, particularly those that are other than a disorder caused at least in part by polyglutamine aggregation. Examples of such neurodegenerative disorders include Alzheimer's, Parkinson's, and ALS. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a neurological disorder, and/or providing a treatment regimen to the subject for a period of greater than one, two, four, six, or twelve months. The method can also include other features described herein.

In another aspect, the invention features a method for treating or preventing a cardiovascular disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a cardiovascular disorder. Exemplary cardiovascular disorders include: cardiac dysrhythmias, chronic congestive heart failure, ischemic stroke, coronary artery disease and cardiomyopathy. In one embodiment, the cardiovascular disorder is an age-associated cardiovascular disorder. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a cardiovascular disorder, and/or providing a treatment regimen to the subject for a period of greater than one, two, four, six, or twelve months. The method can also include other features described herein.

In another aspect, the invention features a method for treating or preventing a metabolic disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a metabolic disorder. Exemplary metabolic disorders include disorders of glucose or insulin metabolism, e.g., diabetes. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a metabolic disorder, and/or providing a treatment regimen to the subject for a period of greater than one, two, four, six, or twelve months. The method can also include other features described herein.

In another aspect, the invention features a method for treating or preventing a dermatological disorder or dermatological tissue condition. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for, or requires prophylaxis for a dermatological disorder or dermatological tissue condition. Exemplary dermatological disorder or dermatological tissue condition disorders include scarring, wrinkling, and so forth. The method can decrease at least one symptom of the disorder. For example, the method includes administering an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a dermatological disorder or dermatological tissue condition, and/or providing a treatment regimen to the subject for a period of greater than one, two, four, six, or twelve months. The method can also include other features described herein.

In another aspect, the invention features a method of treating or ameliorating tissue repair. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. In one embodiment, the subject has, is at risk for, requires treatment for a tissue repair. For example, the subject may have recurrent tissue repair difficulties. Exemplary implementations for tissue repair include wound healing, burns, ulcers (e.g., ulcers in a diabetic, e.g., diabetic foot ulcers), surgical wounds, sores, and abrasions. The method can decrease at least one symptom of the tissue. For example, the method includes administering (e.g., locally or systemically) an effective amount of an agent which modulates the activity of the component of the GH/IGF-1 axis. In one embodiment, the component, is a pre-IGF-1 component, IGF-1, or a post-IGF-1 component. Exemplary pre-IGF-1 components include: GHS-R, GHRH-R, SST-R, GHRH, Ghrelin, and SST. Exemplary post-IGF-1 components include: PI(3) kinase, PTEN phosphatase, PI(3,4)P2, and PI(3,4,5)P3 phosphatidyl inositol kinases, AKT serine/threonine kinase, and a Forkhead transcription factor such as FOXO-1, FOXO-3, or FOXO-4.

In one embodiment, the component is secreted or present at the cell surface (e.g., a receptor). In another embodiment, the component is intracellular. In one embodiment, the component is a core component of the IGF-1 Receptor signalling pathway. In another embodiment, the component is a somatotroph axis signalling pathway component. The agent can be a direct agonist, e.g., of an inhibitory component of the GH/IGF-1 axis, or a direct antagonist, e.g., of a positively acting component of the GH/IGF-1 axis. The method can further include monitoring the subject, e.g., for a symptom of a tissue repair, and/or providing a treatment regimen to the subject for a period of greater than one, two, four, six, or twelve months. The method can also include other features described herein.

Accordingly, the invention also features a method for treating or preventing Amyotrophic Lateral Sclerosis. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for Amyotrophic Lateral Sclerosis.

In one embodiment, the method includes antagonizing a positively acting component of the GH/IGF-1 axis. For example, the method for treating Amyotrophic Lateral Sclerosis includes administering an agent that is a direct antagonist of a positively acting component of the axis to a subject. For example, the method for treating Amyotrophic Lateral Sclerosis includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating Amyotrophic Lateral Sclerosis includes administering an agent that is a direct agonist of an inhibitory component of the axis to a subject. For example, the method for treating Amyotrophic Lateral Sclerosis includes administering an agent that is a direct agonist of SST-R, PTEN or FOXO.

The invention also features a method for treating or preventing type II diabetes. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for type II diabetes.

In one embodiment, the method includes antagonizing a positively acting component of the GH/IGF-1 axis. For example, the method for treating type II diabetes includes administering an agent that is a direct antagonist of a positively acting component of the axis to a subject. For example, the method for treating type II diabetes includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating type II diabetes includes administering an agent that is a direct agonist of an inhibitory component of the axis to a subject. For example, the method for treating type II diabetes includes administering an agent that is a direct agonist of SST-R, PTEN or FOXO.

Accordingly, the invention also features a method for treating or preventing skeletal muscle atrophy. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for skeletal muscle atrophy.

In one embodiment, the method includes antagonizing a positively acting component of the GH/IGF-1 axis. For example, the method for treating skeletal muscle atrophy includes administering an agent that is a direct antagonist of a positively acting component of the axis to a subject. For example, the method for treating skeletal muscle atrophy includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating skeletal muscle atrophy includes administering an agent that is a direct agonist of an inhibitory component of the axis to a subject. For example, the method for treating skeletal muscle atrophy includes administering an agent that is a direct agonist of SST-R, PTEN or FOXO.

Accordingly, the invention also features a method for treating or preventing multiple sclerosis. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for multiple sclerosis.

In one embodiment, the method includes antagonizing a positively acting component of the GH/IGF-1 axis. For example, the method for treating multiple sclerosis includes administering an agent that is a direct antagonist of a positively acting component of the axis to a subject. For example, the method for treating multiple sclerosis includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating multiple sclerosis includes administering an agent that is a direct agonist of an inhibitory component of the axis to a subject. For example, the method for treating multiple sclerosis includes administering an agent that is a direct agonist of SST-R, PTEN or FOXO.

Accordingly, the invention also features a method for treating or preventing age-related macular degeneration. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for age-related macular degeneration.

In one embodiment, the method includes antagonizing a positively acting component of the GH/IGF-1 axis. For example, the method for treating age-related macular degeneration includes administering an agent that is a direct antagonist of a positively acting component of the axis to a subject. For example, the method for treating age-related macular degeneration includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating age-related macular degeneration includes administering an agent that is a direct agonist of an inhibitory component of the axis to a subject. For example, the method for treating age-related macular degeneration includes administering an agent that is a direct agonist of SST-R, PTEN or FOXO.

Accordingly, the invention also features a method for treating or preventing Parkinson's Disease. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for Parkinson's Disease.

In one embodiment, the method includes antagonizing a positively acting component of the GH/IGF-1 axis. For example, the method for treating Parkinson's Disease includes administering an agent that is a direct antagonist of a positively acting component of the axis to a subject. For example, the method for treating Parkinson's Disease includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating Parkinson's Disease includes administering an agent that is a direct agonist of an inhibitory component of the axis to a subject. For example, the method for treating Parkinson's Disease includes administering an agent that is a direct agonist of SST-R, PTEN or FOXO.

Accordingly, the invention also features a method for treating or preventing neuropathy. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for neuropathy.

In one embodiment, the method includes antagonizing a positively acting component of the GH/IGF-1 axis. For example, the method for treating neuropathy includes administering an agent that is a direct antagonist of a positively acting component of the axis to a subject. For example, the method for treating neuropathy includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating neuropathy includes administering an agent that is a direct agonist of an inhibitory component of the axis to a subject. For example, the method for treating neuropathy includes administering an agent that is a direct agonist of SST-R, PTEN or FOXO.

Accordingly, the invention also features a method for treating or preventing Alzheimer's Disease. The method includes: modulating the activity of a component of the GH/IGF-1 axis in a subject (e.g., a human subject, e.g., an adult human subject), which, modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for Alzheimer's Disease.

In one embodiment, the method includes antagonizing a positively acting component of the GH/IGF-1 axis. For example, the method for treating Alzheimer's Disease includes administering an agent that is a direct antagonist of a positively acting component of the axis to a subject. For example, the method for treating Alzheimer's Disease includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating Alzheimer's Disease includes administering an agent that is a direct agonist of an inhibitory component of the axis to a subject. For example, the method for treating Alzheimer's Disease includes administering an agent that is a direct agonist of SST-R, PTEN or FOXO.

Accordingly, the invention also features a method for treating or preventing a neurodegenerative disorder, e.g., other than a neurodegenerative disorder caused at least in part by polyglutamine aggregation. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for a neurodegenerative disorder, e.g., other than a neurodegenerative disorder caused at least in part by polyglutamine aggregation,.

In one embodiment, the method includes antagonizing a positively acting component of the GH/IGF-1 axis. For example, the method for treating a neurodegenerative disorder ncludes administering an agent that is a direct antagonist of a positively acting component of the axis to a subject. For example, the method for treating a neurodegenerative disorder includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating a neurodegenerative disorder includes administering an agent that is a direct agonist of an inhibitory component of the axis to a subject. For example, the method for treating a neurodegenerative disorder, e.g., other than a neurodegenerative disorder caused at least in part by polyglutamine aggregation, includes administering an agent that is a direct agonist of SST-R, PTEN or FOXO.

Accordingly, the invention also features a method for treating or preventing disorder that is caused at least in part by protein misfolding or protein aggregation. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for disorder that is caused at least in part by protein misfolding or protein aggregation.

In one embodiment, the method includes antagonizing a positively acting component of the GH/IGF-1 axis. For example, the method for treating disorder that is caused at least in part by protein misfolding or protein aggregation includes administering an agent that is a direct antagonist of a positively acting component of the axis to a subject. For example, the method for treating disorder that is caused at least in part by protein misfolding or protein aggregation includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating disorder that is caused at least in part by protein misfolding or protein aggregation includes administering an agent that is a direct agonist of an inhibitory component of the axis to a subject. For example, the method for treating disorder that is caused at least in part by protein misfolding or protein aggregation includes administering an agent that is a direct agonist of SST-R, PTEN or FOXO.

Accordingly, the invention also features a method for treating or preventing an inflammatory disorder. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for an inflammatory disorder.

In one embodiment, the method includes antagonizing a positively acting component of the GH/IGF-1 axis. For example, the method for treating an inflammatory disorder includes administering an agent that is a direct antagonist of a positively acting component of the axis to a subject. For example, the method for treating an inflammatory disorder includes administering an agent that is a direct antagonist of GH, IGF-1, GHRH, GHS, and their respective receptors to a subject.

In one embodiment, the method includes agonizing an inhibitory component of the GH/IGF-1 axis to a subject. For example, the method for treating an inflammatory disorder includes administering an agent that is a direct agonist of an inhibitory component of the axis to a subject. For example, the method for treating an inflammatory disorder includes administering an agent that is a direct agonist of SST-R, PTEN or FOXO.

Accordingly, the invention also features a method for treating or preventing breast cancer. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for breast cancer.

In one embodiment, the method includes antagonizing a positively acting component of the GH/IGF-1 axis. For example, the method for treating breast cancer includes administering an agent that is a direct antagonist of a positively acting component of the axis to a subject. For example, the method for treating breast cancer includes administering an agent that is a direct antagonist of GH, IGF-1, GHRH, GHS, and their respective receptors to a subject.

In one embodiment, the method includes agonizing an inhibitory component of the GH/IGF-1 axis to a subject. For example, the method for treating breast cancer includes administering an agent that is a direct agonist of an inhibitory component of the axis to a subject. For example, the method for treating breast cancer includes administering an agent that is a direct agonist of SST-R, PTEN or FOXO.

Accordingly, the invention also features a method for treating or preventing lung cancer. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for lung cancer.

In one embodiment, the method includes antagonizing a positively acting component of the GH/IGF-1 axis. For example, the method for treating lung cancer includes administering an agent that is a direct antagonist of a positively acting component of the axis to a subject. For example, the method for treating lung cancer includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating lung cancer includes administering an agent that is a direct agonist of an inhibitory component of the axis to a subject. For example, the method for treating lung cancer includes administering an agent that is a direct agonist of SST-R, PTEN or FOXO.

Accordingly, the invention also features a method for treating or preventing prostate cancer. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for prostate cancer.

In one embodiment, the method includes antagonizing a positively acting component of the GH/IGF-1 axis. For example, the method for treating prostate cancer includes administering an agent that is a direct antagonist of a positively acting component of the axis to a subject. For example, the method for treating prostate cancer includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating prostate cancer includes administering an agent that is a direct agonist of an inhibitory component of the axis to a subject. For example, the method for treating prostate cancer includes administering an agent that is a direct agonist of SST-R, PTEN or FOXO.

Accordingly, the invention also features a method for treating or preventing colorectal cancer. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for colorectal cancer.

In one embodiment, the method includes antagonizing a positively acting component of the GH/IGF-1 axis. For example, the method for treating colorectal cancer includes administering an agent that is a direct antagonist of a positively acting component of the axis to a subject. For example, the method for treating colorectal cancer includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating colorectal cancer includes administering an agent that is a direct agonist of an inhibitory component of the axis to a subject. For example, the method for treating colorectal cancer includes administering an agent that is a direct agonist of SST-R, PTEN or FOXO.

Accordingly, the invention also features a method for treating or preventing obesity. The method includes: modulating the activity of a component of the GH/IGF-1 axis in a subject (e.g., a human subject, e.g., an adult human subject), which modulation increases Forkhead activity (e.g., FOXO-1, -3, or -4 activity) in a cell of the subject. For example, the method can decrease at least one symptom of the disorder. In one embodiment, the subject has, is at risk for, requires treatment for, or requires prophylaxis for obesity.

In one embodiment, the method includes antagonizing a positively acting component of the GH/IGF-1 axis. For example, the method for treating obesity includes administering an agent that is a direct antagonist of a positively acting component of the axis to a subject. For example, the method for treating obesity includes administering an agent that is a direct antagonist of GH, IGF-1, GHRH, GHS, or their respective receptors to a subject.

In one embodiment, the method includes agonizing an inhibitory component of the GH/IGF-1 axis to a subject. For example, the method for treating obesity includes administering an agent that is a direct agonist of an inhibitory component of the axis to a subject. For example, the method for treating obesity includes administering an agent that is a direct agonist of SST-R, PTEN or FOXO.

In another aspect, the invention provides a method of modulating lifespan regulation or potential n a subject. The method includes: a) evaluating an indicator of GH/IGF-1 axis activity in an adult subject that does not have acromegaly or supranormal levels of GH or IGF-1 with respect to age; and b) administering, to the subject, a regimen of doses of a compound that alters (e.g., inhibits or enhances) activity of a GH/IGF-1 axis component, the regimen being a function of the indicator and effective to maintain detectable, subnormal levels of IGF-1 in the subject with respect to age. Exemplary indicators of GH/IGF-1 axis activity include a parameter (e.g., concentration) that is a function of circulating hormone levels (e.g., GH or IGF-1), intracellular signaling, pituitary or hypothalamus physiology, and so forth. In a related method, an age-associated parameter or a parameter that is a function of caloric restriction is evaluated.

In another aspect the invention provides a method of evaluating a compound for a modulatory effect on life span regulation. The method includes: a) providing a test compound; b) contacting the test compound to a GH/IGF-1 axis component in vitro; c) evaluating interaction between the test compound and the growth hormone/IGF-1 axis component; and d) evaluating a parameter (e.g., a GH/IGF-1 axis associated parameter, an age-associated parameter; a marker of caloric restriction) of an adult subject (e.g., a mammal, e.g., a human, e.g., a non-human primate) treated with the test compound, wherein an interaction between the test compound, the GH/IGF-1 axis component, and modulation of the parameter relative to a control subject identifies the respective compound as having a modulatory effect on lifespan regulation. Exemplary parameters are described herein. The method can be implemented using a cohort of animals (e.g., non-human animals, e.g., mammals, e.g., rats, mice, primates, cows, pigs, and so forth). The animal can be an animal with a disease or disorder, e.g., an age related disease or disorder.

In another aspect, the method of evaluating a compound for a modulatory effect on life span regulation includes: a) providing a test compound; b) contacting the test compound to a GH/IGF-1 axis component in vitro; c) evaluating interaction between the test compound and the growth hormone/IGF-1 axis component; and d) evaluating a parameter of the cell, for example, an age-associated parameter of a cell (e.g., a fibroblast, an osteoblast, a skin cell, a blood cell, a transformed cell, a senescent cell, a cultured cell, or a neural cell) treated with the test compound, wherein an interaction between the test compound the GH/IGF-1 axis component and modulation of the age-associated parameter relative to a control cell identifies the respective compound as having a modulatory effect on lifespan regulation. In one embodiment, the age-associated parameter includes one or more of:

(i) lifespan of the cell or the organism;

(ii) presence or abundance of a gene transcript or gene product in the cell or organism that has a biological age-dependent expression pattern;

(iii) resistance of the cell or organism to stress;

(iv) one or more metabolic parameters of the cell or organism;

(v) proliferative capacity of the cell or a set of cells present in the organism; and

(vi) physical appearance or behavior of the cell or organism. In another embodiment, the in vitro contacting is a cell-based assay or a cell-free assay. In yet another embodiment, the adult subject has normal IGF-1 levels. In yet another embodiment, the evaluating of interaction with the GH/IGF-1 axis component includes measuring levels of GH or IGF-1. In yet another embodiment, the GH/IGF-1 axis component is a cell surface receptor component. In still another embodiment, the evaluating of interaction with the GH/IGF-1 axis component includes evaluating levels or activity of a component downstream of the component that is being targeted. For example, the component can be a post-IGF-1 component or a IGF-1 Receptor signalling pathway effector. The method can include other features described herein.

In another aspect, the invention provides a method of evaluating a compound for a modulatory effect on life span regulation. The method includes: a) providing a library of multiple compounds, b) contacting each compound of the library to a GH/IGF-1 axis component in vitro; c) evaluating interaction between each compound and the GH/IGF-1 axis component; d) selecting a subset of compounds from the library based on the evaluated interactions; e) administering (e.g., individually) one or more compounds of the subset to an adult subject (e.g., a mammal, e.g., a human, e.g., a non-human primate, ); and f) evaluating an age-associated parameter of the adult subject, wherein modulation of the age-associated parameter relative to a control subject identifies the respective compound as having a modulatory effect on lifespan regulation.

The library can include at least 50, 10³, 10⁵, 10⁶, or 10⁸ compounds, e.g., between 10³ and 10⁷ compounds. In one embodiment, the compounds can be less than 6, 5, 4, 3, 2, 1, 0.7, 0.6, 0.5, 0.4, 0.3, or 0.2 kDa. In another embodiment, each compound can be a protein.

In one embodiment, the library includes one or more of an immunoglobulin, a peptide, a nucleic acid aptamer, a dsRNA, a siRNA, a ribozyme, or an antisense nucleic acid. In one embodiment, the library is a display library. For example, each compound of the library is displayed on the surface of cell or virus. In one embodiment, each compound of the library or a subset thereof is encoded by a nucleic acid. In one embodiment, each compound of the library or a subset thereof is non-polymeric. In one embodiment, each compound of the library is substantially free of a nucleic acid polymer or peptide. In one embodiment, the library includes a plurality of diverse peptides.

In one embodiment, the age-associated parameter includes one or more of: (i) lifespan of the cell or the organism; (ii) presence or abundance of a gene transcript or gene product in the cell or organism that has a biological age-dependent expression pattern; (iii) resistance of the cell or organism to stress (e.g., oxidative or genotoxic stress, starvation); (iv) one or more metabolic parameters of the cell or organism; (v) proliferative capacity of the cell or a set of cells present in the organism; and (vi) physical appearance or behavior of the cell or organism. In another embodiment, the in vitro contacting is a cell-based assay or a cell-free assay. In yet another embodiment, the adult subject has normal IGF-1 levels. In yet another embodiment, the evaluating of interaction with the GH/IGF-1 axis component includes measuring levels of GH or IGF-1. In yet another embodiment, the GH/IGF-1 axis component is a cell surface receptor component. Evaluation of an interaction can refer to evaluating a binding interaction or an enzymatic interaction (e.g., modification, e.g., phosphorylation, of a substrate).

In one embodiment, the library includes multiple different spiropiperidine molecules (e.g. MK0677-like) or multiple different benzo-fused lactam molecules (e.g. L-739,943-like).

A further embodiment includes synthesizing a second library of compounds that include a set of features of a compound of the subset; and repeating the method. In another further embodiment the method includes formulating an identified compound as a pharmaceutical composition. The method can include other features described herein.

In another aspect, the invention features a method of evaluating a compound for a modulatory effect on a disorder. The method includes: a) providing a library of compounds; b) contacting each compound of the library to a GH/IGF-1 axis component in vitro; c) evaluating interaction between each compound and the GH/IGF-1 axis component; d) selecting a subset of compounds from the library based on the evaluated interactions; e) contacting a compound of the subset to (i) a cell in vitro, the cell being from a subject having the disorder or from non-human animal model of the disorder, or (ii) a non-human animal model of the disorder; and f) evaluating the cell or the animal model, wherein a change in an parameter of the disorder identifies the respective compound as having a modulatory effect on the disorder. In one embodiment, contacting the compound to the animal model comprises administering the compound to the animal model. The disorder can be, e.g., a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardio-vascular disorder. The component can be a post-IGF1 component, IGF-1, or a pre-IGF1 component. The in vitro contacting can be to a cell that includes the component (e.g., as a recombinant or exogenous protein) or can be a cell-free system, e.g., a system in which the component is at least partially purified. The method can include other features described herein.

In another aspect, the invention features a method of evaluating a compound for a modulatory effect on a disorder. The method includes: a) selecting a GH/IGF-1 axis modulator; b) contacting the modulator to (i) a cell in vitro, the cell being from a subject having the disorder or from non-human animal model of the disorder, or (ii) a non-human animal model of the disorder; and c) evaluating the cell or the animal model, wherein a change in an parameter of the disorder identifies the respective compound as having a modulatory effect on the disorder. The disorder can be, e.g., a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardiovascular disorder. In one embodiment, the modulator is a compound that directly antagonizes a positively acting GH/IGF-1 axis component. In another embodiment, the modulator is a compound that directly agonizes an inhibitory GH/IGF-1 axis component. The method can include other features described herein.

In another aspect, the invention provides a method of evaluating a compound for a modulatory effect on life span regulation. The method includes: a) administering a test compound to an adult mammal in a regimen that extends for at least a month; b) evaluating a GH/IGF-1 axis component in the adult mammal at least once during the regimen; and c) evaluating an age-associated parameter of the adult mammal at least after a month months (e.g., 3, 6, 9, or 12 months or for 1, 2, 5, 10, 20, or 30 years) of the regimen, wherein modulation of the age-associated parameter relative to that of a control subject identifies the test compound as having a modulatory effect on lifespan regulation. The method can include other features described herein.

In another aspect, the invention provides a method of identifying a GH/IGF-1 axis antagonist or partial agonist. The method includes: a) providing a test compound that is obtained by chemically modifying an agonist of a GH/IGF-1 axis component or that is selected for structural similarity to an agonist of a GH/IGF-1 axis component; and b) evaluating a property (e.g., level, activity, or downstream signaling) of a GH/IGF-1 axis component in vitro, in a cell, or in an organism in the presence of the test compound, wherein alteration (e.g., antagonism or partial agonism) of the property of the GH/IGF-1 axis component identifies the test compound as a GH/IGF-1 axis antagonist.

In one embodiment, the compound is a spiropiperidine molecule (e.g., MK0677-like) or a benzo-fused lactam molecule (e.g., L-739,943-like). The method can include other features described herein.

In another aspect the invention provides a method of identifying a compound that decreases senescence that includes: a) providing a test compound that is obtained by chemically modifying an agonist of a GH/IGF-1 axis component (e.g., to impart antagonist or partial agonist properties) or that is selected for structural similarity to an agonist of a GH/IGF-1 axis component; b) administering the test compound to an adult subject; and c) evaluating levels of a GH/IGF-1 axis component, wherein decreased levels of growth hormone and/or IGF-1 identifies the test compound as an agent that decreases senescence.

In one embodiment, the compound is a spiropiperidine molecule (e.g., MK0677-like) or a benzo-fused lactam molecule (e.g., L-739,943-like). A further embodiment includes d) evaluating an age-associated parameter of a subject treated with the test compound, wherein modulating the age-associated parameter relative to a control subject further identifies the test compound as an agent that decreases senescence.

In another embodiment, the subject is an adult with normal IGF-1 levels. The adult subject can be a member of a cohort of adult subjects that are treated and evaluated. Each subject of the cohort can be characterized by normal IGF-1 levels prior to treatment. The method can include other features described herein.

In another aspect, the invention provides a method of identifying an agent that modulates lifespan regulation of an adult animal. The method includes: a) selecting an agent that alters a property of the GH/IGF-1 axis; b) administering the agent to a subject; and c) evaluating an age-associated parameter in the subject, wherein modulation of the age-associated parameter identifies the agent as an agent that modulates lifespan regulation.

In one embodiment, the agent is an antagonist of a component of the GH/IGF-1 axis. In another embodiment, the agent decreases GH secretion. In yet another embodiment, the agent partially blocks GH binding to GH receptor. In yet another embodiment, the agent decreases IGF-1 serum levels, blocks GH activation of IGF-1 secretion, or blocks a GH secretagogue receptor activity. In yet another embodiment, the agent activates or increases levels of somatostatin. In another embodiment, the agent activates or increases the levels of somatostatin receptor. The method can include other features described herein.

In another aspect, the invention provides a method of identifying an agent that modulates lifespan regulation of an adult animal. The method includes: a) selecting an agent that alters a property of the GH/IGF-1 axis; b) administering the agent to a subject, wherein the subject has an age-related disorder; and c) evaluating a parameter of the subject.

In one embodiment, the parameter is an indicator of the severity of the disorder. The disorder can be a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardio-vascular disorder. The method can include other features described herein.

In another aspect, the invention includes administering an agent that is an agonist of the GH/IGF-1 axis to a subject, the agent being administered chronically and in an amount effective to cause overall down-regulation, e.g., by triggering a negative feedback loop. For example, the agonist can be a GHS agonist or a super-agonist of a positively acting axis component. The method can include other features described herein.

In still another aspect, a partial agonist of the GH/IGF-1 axis is administered to a subject in an amount effective to downregulate activity of the axis. For example, the partial agonist can decrease physiological signaling of certain positively acting components of the axis (such as growth hormone secretagogue), if the partial agonist has a higher potency than the physiological secretagogue, yielding the net effect of overall downregulation of the axis. The method can include other features described herein.

The invention features, inter alia, methods for treating or preventing neurodegenerative disorders and disorders that are caused at least in part by protein misfolding or aggregation, e.g., aggregation other than by polyglutamine aggregation. One exemplary neurodegenerative disorder that is caused at least in part by protein aggregation is Alzheimer's Disease or Parkinson's Disease.

In one aspect, the invention features a method of treating or preventing a disorder, e.g., a neurodegenerative disorder or a disorder caused by protein misfolding or aggregation in a subject. The method includes reducing activity of the IGF-1/GH axis in the subject. For example, the subject is a mammal, particularly a human. In one embodiment, the neurodegenerative disorder is not caused by aggregation of a polyglutamine protein.

In a particular embodiment, the disorder is Alzheimer's disease or Parkinson's disease.

In one embodiment, the method includes administering a composition that reduces IGF-1/GH axis activity. Typically, the composition is administered in an amount effective to reduce or prevent at least one symptom of the disorder (e.g., a clinical symptom) or in an amount effective to reduce or prevent the disorder. In one embodiment, the composition includes an agonist of an inhibitory component of the IGF-1/GH axis. For example, the inhibitory component of the IGF-1/GH axis is a somatostatin receptor (SST2 or SST5), a PTEN phosphatase, or a FOXO transcription factor (e.g., Forkhead). Exemplary agonists for inhibitory components include somatostatin, L-054,522, BIM-23244, BIM-23197, BIM-23268, octreotide, TT-232, butreotide, lanreotide, or vapreotide, as well as others described herein and that those that can be identified by the methods described herein.

In another embodiment, the composition includes an antagonist of an activator of the IGF-1/GH axis or a component that promotes or is required for an activity of the IGF-1/GH axis. For example, the component of the IGF-1/GH axis is GH, GHRH, GHRH-R, GHS, GHS-R, GH-R, PI-3 kinase, PDK-1, or an AKT kinase. In one embodiment, the antagonist is a kinase inhibitor. In another embodiment, the antagonist is an antibody to a hormone (e.g., GH, GHS, or GHRH) or an antibody or other agent that binds to a cell surface receptor (e.g., GH-R, GHRH-R, or GHS-R). Functional antibody fragments can also be used. In one embodiment, the antagonist is a modified ligand of the cell surface receptor. For example, the antagonist is a modified growth hormone molecule that antagonizes GH-R, e.g., Pegvisomant.

An exemplary antagonist of GHS or the GHS-R is a modified peptide, e.g., [D-Lys³]-GHRP-6.

In another embodiment, the composition includes a compound that is a dopamine agonist that decreases GH production.

In one embodiment, the composition includes an agent described herein, e.g., listed in Table 2.

Generally, a compound in the composition that modulates GH/IGF-1 axis activity can be a small organic molecule (e.g., less than 7 kDa in molecular weight, e.g., 6, 5, 4, 3, 2, 1, or 0.5 kDa). The compound can also be a peptide, polypeptide, antibody, antibody fragment, peptidomimetic, peptoid, nucleic acid, or other chemical compound or a combination of any of these.

In one embodiment, the composition is administered at regular intervals (e.g., daily, weekly, biweekly, or monthly). In yet another embodiment, the composition is administered at regular intervals for at least two months (e.g., preferably, at least six or nine months or for at least one, two, five, ten, 20, 25, or 30 years).

In an embodiment, the method further includes, e.g., prior to the reducing the activity of the IGF-1/GH axis, identifying the subject as a subject having or predisposed to having the disorder.

In another aspect, the invention features a method of treating a subject. The method includes: identifying a mammalian subject as having or being disposed to having a disorder caused at least in part by protein misfolding or aggregation , e.g., aggregation other than by polyglutamine aggregation.; and providing a treatment to the subject, wherein the treatment antagonizes activity of the IGF-1/GH axis in the subject. The treatment can be prophylactic or provided as a curative (e.g., after the onset of at least one symptom). For example, the subject is a mammal, particularly a human. In one embodiment, the neurodegenerative disorder is not caused by aggregation of a polyglutamine protein. One exemplary neurodegenerative disorder that is caused at least in part by protein aggregation is Alzheimer's Disease or Parkinson's Disease.

In one embodiment, the treatment includes administering a composition that reduces IGF-1/GH axis activity.

Typically, the composition is administered in an amount effective to reduce or prevent at least one symptom of the disorder (e.g., a clinical symptom) or in an amount effective to reduce or prevent protein misfolding or aggregation. The pharmaceutically “effective amount” for purposes herein is determined by such considerations as are known in the art. The amount is effective either to achieve improvement in at least one clinical signs and/or symptoms—including but not limited to decreased levels of protein misfolding or aggregation (e.g., decreased formation of amyloid or Lewy bodies), or improvement or elimination of symptoms and other clinical endpoints—or to delay onset of or progression of signs or symptoms of disease, as are selected as appropriate clinical indicia. Cure is not required, nor is it required that improvement or delay, as above described, be achievable in a single dose.

In one embodiment, the treatment is sufficient to reduce levels of GH, levels of IGF-1, levels of IGF-1 receptor signalling in the subject by at least 30% (e.g., at least 50, 60, 70, or 80%) of a normal level for the chronological age of the adult subject, but not below detection. The reduction can include reducing the level to a resulting level that is less than 90, 80, 70, 60, 50, or 30% and/or greater than 70, 65, 60, 55, 50, 45, 40, or 15% of the initial level of the subject. In another example, partial reduction can include reducing a level to a resulting level that is less than 90, 80, 70, 60, 50, or 30% and/or greater than 70, 65, 60, 55, 50, 45, 40, or 15% of the average level among normal individuals having the same age and gender as the subject.

Treatment can be commenced at least prior to clinical onset of the disorder or provided at least at some point after clinical onset of the disorder or onset of at least one symptom (e.g., clinical symptom) of the disorder.

In one embodiment, the subject is an adult. Typically, the subject is an adult (e.g., a human adult having an age of at least 18, 21, 24, or 28 years) without defects in the GH/IGF-1 axis, and thus does not have acromegaly or diabetic retinopathy. In one embodiment, the adult is at least middle aged, e.g., at least 50, 60, 65, 70, 75, or 80 years of age.

In one embodiment, the composition includes an agonist of an inhibitory component of the IGF-1/GH axis. For example, the inhibitory component of the IGF-1/GH axis is a somatostatin receptor (SST2 or SST5), a PTEN transcription factor, or a FOXO transcription factor (e.g., Forkhead). Exemplary agonists for inhibitory components include somatostatin, L-054,522, BIM-23244, BIM-23197, BIM-23268, octreotide, TT-232, butreotide, lanreotide, or vapreotide, as well as others described herein and that those that can be identified by the methods described herein.

In another embodiment, the composition includes an antagonist of an activator of the IGF-1/GH axis or a component that promotes or is required for an activity of the IGF-1/GH axis. For example, the component of the IGF-1/GH axis is GH, GHRH, GHRH-R, GHS, GHS-R, GH-R, PI-3 kinase, PDK-1, or an AKT kinase. In one embodiment, the antagonist is a kinase inhibitor. In another embodiment, the antagonist is an antibody to a hormone (e.g., GH, GHS, or GHRH) or an antibody or other agent that binds to a cell surface receptor (e.g., GH-R, GHRH-R, or GHS-R). Functional antibody fragments can also be used. In one embodiment, the antagonist is a modified ligand of the cell surface receptor. For example, the antagonist is a modified growth hormone molecule that antagonizes GH-R, e.g., Pegvisomant.

An exemplary antagonist of GHS or the GHS-R is a modified peptide, e.g., [D-Lys³]-GHRP-6.

In another embodiment, the composition includes compound that is a dopamine agonist that decreases GH production.

In one embodiment, the composition includes an agent described herein, e.g., listed in Table 2.

Generally, a compound in the composition that modulates GH/IGF-1 axis activity can be a small organic molecule (e.g., less than 7 kDa in molecular weight, e.g., e.g., 6, 5, 4, 3, 2, 1, 0.7, 0.6, 0.5, 0.4, 0.3, or 0.2 kDa). The compound can also be a peptide, polypeptide, antibody, antibody fragment, peptidomimetic, peptoid, nucleic acid, or other chemical compound or a combination of any of these.

In one embodiment, the composition is administered at regular intervals (e.g., daily, weekly, biweekly, or monthly). In yet another embodiment, the composition is administered at regular intervals for at least two months (e.g., preferably, at least six or nine months or for at least one, two, five, ten, 20, 25, or 30 years).

In one embodiment, the method further includes monitoring the subject, e.g., for a symptom of the disorder, e.g., for a neurological, anatomical, or biochemical symptom, before, during, and/or after the reducing the activity of the GH/IGF-1 axis. In one embodiment, the monitoring includes imaging neuronal tissue (e.g., at least a part of the brain) of the subject. Images can be evaluated for indications of neuronal cell death, brain lesions, anomalous white matter, neurofibrillary tangles, amyloid deposits, and protein aggregates.

In one embodiment, the monitoring includes a neurological exam (e.g., a cognitive exam, reflex test) or one or more subsections of a standardize rating scale. In one embodiment, the subject is monitored for a parameter of the IGF-1/GH axis.

In one embodiment, the identifying includes evaluating the identity of at least one nucleotide of a gene (or mRNA) of a subject. In an embodiment, the identifying includes evaluating a genetic relative of a subject for a symptom of a neurodegenerative disorder or a symptom of protein misfolding or aggregation.

In one embodiment, the method includes evaluating an indicator of GH/IGF-1 axis activity in the subject and b) administering, to the subject, a regimen of doses of a compound that alters activity of a GH/IGF-1 axis component. The regimen is a function of the indicator and can be effective to maintain detectable, subnormal levels of IGF-1 in the subject with respect to age. Exemplary indicators of GH/IGF-1 axis activity include a parameter (e.g., concentration) that is a function of circulating hormone levels (e.g., GH or IGF-1), intracellular signaling, pituitary or hypothalamus physiology, and so forth. In a related method, an age-associated parameter or a parameter that is a function of caloric restriction is evaluated.

In another aspect, the invention features a kit that includes an active agent that antagonizes the IGF-1/GH pathway and instructions for or administering the agent to treat or prevent a neurodegenerative disorder or a disorder caused at least in part by aggregation (e.g., other than polyglutamine aggregation) or misfolding. The agent can be an agent described herein.

In another aspect, the invention features a labeled container that includes a pharmaceutical composition that includes an active agent that antagonizes the IGF-1/GH pathway, wherein the container includes information for administering the composition to treat or prevent a neurodegenerative disorder or a disorder caused at least in part by aggregation (e.g., other than polyglutamine aggregation) or misfolding..

In another aspect, the invention features a method of evaluating a compound for ability to modulate protein aggregation or misfolding in a cell. The method includes: a) providing a test compound; b) contacting the test compound to a GH/IGF-1 axis component in vitro; c) evaluating interaction between the test compound and the growth hormone/IGF-1 axis component; d) contacting the test compound to a cell; and e) evaluating protein aggregation or misfodling in or around the cell or evaluating the cell for a cellular symptom of aggregation or misfolding. Typically the protein has fewer than 35 or 30 polyglutamine repeats, i.e., it does not have a polyglutamine region.

A related method includes: a) providing a library of compounds, the library including multiple compounds; b) contacting each compound of the library to a GH/IGF-1 axis component in vitro; c) evaluating interaction between each compound and the GH/IGF-1 axis component; d) selecting a subset of compounds from the library based on the evaluated interactions; and e) for each compound of the subset, contacting the compound to a cell, and evaluating aggregation or misfolding in or around the cell or evaluating the cell for a cellular symptom of protein aggregation or misfolding. Typically the cell does not express or include a gene that encodes a protein that has a polyglutamine region.

In one embodiment, the cell is a eukaryotic (e.g., mammalian) cell. For example, the cell expresses a heterologous protein that includes a region of human protein that is protein to aggregation or misfolding, e.g., APP. In one embodiment, the heterologous protein includes a fluorophore (e.g., the protein is a fluorescent protein, e.g., GFP, YFP, etc.).

In one embodiment, the cellular symptom of misfolding or aggregation includes expression and/or subcellular localization of a heat shock protein. For example, the evaluating includes photobleaching and evaluating recovery of fluorescence after photobleaching.

The method can also include evaluating a parameter of the cell, in addition to protein misfolding or aggregation, for example, an age-associated parameter of a cell (e.g., a neuronal cell, a fibroblast, an osteoblast, a skin cell, a blood cell, a transformed cell, a senescent cell, or any cultured cell) treated with the test compound. In one embodiment, the age-associated parameter includes one or more of: (i) lifespan of the cell or the organism; (ii) presence or abundance of a gene transcript or gene product in the cell or organism that has a biological age-dependent expression pattern; (iii) resistance of the cell or organism to stress; (iv) one or more metabolic parameters of the cell or organism; (v) proliferative capacity of the cell or a set of cells present in the organism; and (vi) physical appearance or behavior of the cell or organism. In another embodiment, the in vitro contacting is a cell-based assay or a cell-free assay.

Still another related method includes: a) providing a test compound; b) contacting the test compound to a GH/IGF-1 axis component in vitro; c) evaluating interaction between the test compound and the growth hormone/IGF-1 axis component; d) administering the test compound to a subject organism; and e) evaluating protein misfolding or aggregation in the subject organism, a symptom of misfolding or aggregation, or a neurological symptom. Typically, the organism does not include a cell that expresses a protein that has a polyglutamine region. For example, the organism does not include a cell include a gene that encodes such a protein.

Still another related method includes: a) providing a library of compounds, the library including multiple compounds; b) contacting each compound of the library to a GH/IGF-1 axis component in vitro; c) evaluating interaction between each compound and the GH/IGF-1 axis component; d) selecting a subset of compounds from the library based on the evaluated interactions; and e) for each compound of the subset, administering the compound to a subject organism, and evaluating the subject organism for protein misfolding or aggregation, a symptom of misfolding or aggregation, or a neurological symptom.

In one embodiment, the organism is an invertebrate organism. In another embodiment, the organism is a vertebrate organism, e.g., a non-human mammal. The organism can include cells containing a heterologous nucleic acid encoding a protein that is predisposed to aggregation, e.g., a protein that can form amyloids (e.g., amyloid deposits) or plaques. The heterologous nucleic acid can be a transgene or extrachromosomal element. The method can be implemented using a cohort of organisms (e.g., non-human animals, e.g., mammals, e.g., rats, mice, primates, cows, pigs, and so forth). Statistics may be used to evaluate the cohort of organisms, e.g., to detect a statistically significant effect.

In one embodiment, the protein does not include a polyglutamine region. In another embodiment, the cell expresses an endogenous protein that is prone to aggregation. For example, the heterologous protein includes a fluorophore (e.g., the protein is a fluorescent protein, e.g., GFP, YFP, etc.).

The library can include at least 50, 10³, 10⁵, 10⁶, or 10⁸ compounds, e.g., between 10³ and 10⁷ compounds. The compounds can be less than 100 000, 60 000, or 30 000 Daltons. In another embodiment, the compounds can be less than 7000, 5000, or 3000 Daltons. In one embodiment, the library of compounds includes at least 50, 10³, 10⁵, 10⁶, or 10⁸ structurally related compounds, e.g., derivatives of a compound described herein. In one embodiment, the library includes a collection of naturally occurring compounds. In another embodiment, the library includes a collection of artificial compounds. The library can be a library of proteins, of nucleic acids (e.g., siRNAs), or precursors thereof (e.g., a library of nucleic acids that can be expressed to produce a library of proteins or that can be processed or transcribed to produce double-stranded RNAs (e.g., siRNAs)).

In one embodiment, the library includes multiple different spiropiperidine molecules (e.g. MK0677-like) or multiple different benzo-fused lactam molecules (e.g. L-739,943-like).

A further embodiment includes synthesizing a second library of compounds that include a set of features of a compound of the subset; and repeating the method. In another further embodiment the method includes formulating an identified compound as a pharmaceutical composition. The method can include other features described herein.

In another aspect, the invention features a method for gathering genetic information, the method including: a) determining the identity of at least one nucleotide in gene encoding an IGF-1/GH axis component of a human subject; and b) creating a record which includes information about the identity of the nucleotide and information relating to a neurodegenerative disorder-related parameter from an evaluation of the subject.

A related method includes a) determining the identity of at least one nucleotide in gene encoding an IGF-1/GH axis component for a plurality of subjects who have a particular disorder (e.g., a neurodegenerative disorder or a disorder associated with protein misfolding or aggregation, e.g., other than polyglutamine aggregation) or are associated with such a particular disorder; and b) evaluating the distribution of one or more nucleotide identities for a given position in the gene among or between subjects of the plurality.

The invention also features a computer-readable database that includes a plurality of records, each record including: a) a first field which includes information about one or more nucleotides from a gene encoding an IGF-1/GH axis component of a subject and; b) a second field which includes information about a phenotype of the subject, wherein the phenotype is associated with a neurodegenerative disorder or a disorder associated with protein misfolding or aggregation, e.g., other than polyglutamine aggregation. The information about the phenotype can include information about a biochemical parameter of the subject, anatomical parameter of the subject, or cognitive parameter of the subject. The information about the phenotype can include a diagnosis, e.g., a diagnosis of the disorder.

In another aspect, the invention features a method for gathering genetic information, the method including: a) determining the identity of at least one nucleotide in gene encoding an IGF-1/GH axis component of a human subject; and b) creating a record which includes information about the identity of the nucleotide and information relating to an age-related parameter or a parameter about an age-related disorder (e.g., about a symptom of an age-related disease) from an evaluation of the subject.

A related method includes a) determining the identity of at least one nucleotide in gene encoding an IGF-1/GH axis component for a plurality of subjects who have survived to a predetermined age, e.g., old age, e.g., at least 70, 80, 90, 95, 97, or 99 percentile for the population in which they are found, e.g., at least 75, 80, 85, 90, 95, 97, 98, 100, or 105 years of age; and b) evaluating the distribution of one or more nucleotide identities for a given position in the gene among or between subjects of the plurality.

In one embodiment, the age-related parameter is a biochemical parameter, e.g., an assessment of gene or protein expression. For example, the parameter can relate to an protein associated with old age or an age-related disease (e.g., a cancer specific antigen, an amyloid protein, protein aggregate or misfolded structure, growth hormone, insulin, IGF-1, Ab42, or tau). Other examples include non-protein components, e.g., metabolites, cofactors (such as vitamin B12) and nutrients. For example, the assessment can be of blood, plasma, serum, cerebrospinal fluid (CSF), a biopsy, urine, skin, and so forth. In another embodiment, the age-related parameter is an assessment of neurological function, e.g., cognitive function, motor control, reflex speed, etc. The age-related parameter includes a result of a mental examination, a memory test, a behavioral test, a personality test, or other cognitive test. For example, the age-related parameter includes information about a symptom of dementia. For example, the symptom of dementia includes at least one of the following: decline in mental status; loss of recent memory; inability to learn and remember new information; behavioral disorganization; diminished abstract thinking; diminished judgment; and personality changes (e.g., mood swings, irritability).

In one embodiment, the age-related parameter is an anatomical feature, e.g., a feature of the brain, cardiovascular symptom, or a tumor. Exemplary methods for evaluating anatomical features include radiological methods, such as X-ray, and multi-dimensional imaging techniques such as MRI or computed tomography.

In another example, the age-related parameter includes information about a genetic polymorphism associated with age-associated disease.

The invention also features a computer-readable database that includes a plurality of records, each record including: a) a first field which includes information about one or more nucleotides from a gene encoding an IGF-1/GH axis component of a subject and; b) a second field which includes information about a phenotype of the subject, wherein the phenotype is associated with age, aging, or an age-related disorder. The information about the phenotype can include information about a biochemical parameter of the subject, anatomical parameter of the subject, or cognitive parameter of the subject. The information about the phenotype can include a diagnosis, e.g., a diagnosis of an age-related disorder.

Growth hormone (GH) is a 22 kDa, 191 amino acid single chain peptide containing two disulfide bridges. In humans, GH is essential for linear growth of the infant, child, and adolescent and also plays an important role in the regulation of metabolism. In mammals, it is the primary hormone responsible for growth, and it accelerates metabolic processes such as lipolysis and protein synthesis. GH and many other hormones are part of a complex endocrine system, called the GH/insulin-like growth factor-1 axis (GH/IGF-1 axis).

GH secretion and circulating IGF-1 levels are regulated by the GH/IGF-1 axis. Included in the GH/IGF-1 axis are hormones from the hypothalamus and from elsewhere in the body, receptors on the anterior pituitary and peripheral tissues and organs, anterior pituitary somatotrophs that produce and secrete GH, and peripheral tissues that secrete IGF-1 in response to GH. FIG. 6 is a schematic of the GH/IGF-1 axis.

GH secretion occurs in a pulsatile manner due to the action of both positive and negative regulation originating from the hypothalamus. The hypothalamic peptide, GH releasing hormone (GHRH), and the endogenous GH secretagogue (GHS), ghrelin, are positive regulators of GH and act on the hypothalamus and/or anterior pituitary somatotrophs (cells that produce GH) to release GH. Human GHRH is a C-amidated 44 amino acid peptide. It is present and secreted from the hypothalamus. GHRH binds to specific GHRH receptors on the anterior pituitary thus causing GH release by the anterior pituitary somatotroph. Somatostatin, on the other hand, opposes the action of GHRH and ghrelin by blocking GH release. Somatostatin is a fourteen amino acid peptide that includes a cyclic loop bound by a disulfide bridge. Equally active synthetic versions of somatostatin can be in the reduced or linear state. Somatostatin is found in high concentrations in the hypothalamus, is produced by a large number of tissues, and participates in a wide array of biological functions, including decreasing GH release. Many neurotransmitters and neuropeptides are also involved in the control of GH secretion with both stimulatory and inhibitory effects, e.g., via interaction with GHRH and somatostatin.

The GH/IGF-1 axis includes a series of extracellular and intracellular signalling components that have as a downstream target, the transcription factor Forkhead. Major components of the GH/IGF-1 axis are shown in FIG. 2. The components can be divided into three categories: pre-IGF-1, IGF-1, and post-IGF-1 components. “Pre-IGF-1 components” include GH, GHS, GHS-R, GHRH, GHRH-R, SST, and SSTR. “Post-IGF-1 components” include IGF-1-R and intracellular signalling components including PI(3) kinase, PTEN phosphatase, PI(3,4)P₂, 14-3-3 protein, and PI(3,4,5)P₃ phosphatidyl inositol kinases, AKT serine/threonine kinase (e.g., AKT-1, AKT-2, or AKT-3), or a Forkhead transcription factor (such as FOXO-1, FOXO-3, or FOXO-4).

A “core component of the IGF-1 Receptor signalling pathway” refers to a component that is one of the following: (i) the IGF-1 receptor (IGF1R), (ii) a Forkhead transcription factor that responds to IGF1R signalling, or (iii) a protein that participates in signal transduction between IGF1R and the Forkhead transcription factor. Examples of proteins that participate in this signal transduction include PI(3) kinase, PTEN phosphatase, PI(3,4)P2, and PI(3,4,5)P3 phosphatidyl inositol kinases, PDK-1 (3-phosphoinositide-dependent kinase-1), and AKT serine/threonine kinase (e.g., AKT-1, AKT-2, or AKT-3).

A “somatotroph axis signalling pathway component” refers to a protein that is one of the following: (i) a protein that is located in a somatotroph and that regulates GH release by the somatotroph, or (ii) a protein that directly binds to a protein in class (i). Exemplary somatotroph axis signalling pathway components of class (i) include cell surface receptors such as GHS-R, GHRH-R, and SST-R. Exemplary somatotroph axis signalling pathway components of class (ii) include GHRH, Ghrelin, and SST.

A “IGF-1 Receptor signalling pathway effector” refers a protein or other biologic whose levels are directly regulated by a Forkhead transcription factor in response to IGF-1. For example, expression of the gene encoding the protein can be directly regulated by a Forkhead transcription factor such as FOXO-1, FOXO-2, or FOXO-3. Exemplary IGF-1 Receptor signalling pathway effector can include: GADD45, PA26, Selonoprotein P, Whip1, cyclin G2, and NIP3.

An “inhibitory component of the axis” is a component whose wild-type activity is to decrease axis activity. Exemplary inhibitory components include somatostatin, PTEN, and Forkhead. A “positively acting component of the axis” is a component whose wild-type activity contributes to axis activity. Exemplary inhibitory components include GHRG, GHS, GH, IGF-1, their respective receptors, PI(3) Kinase, PDK-1 and AKT.

In one embodiment, the GH/IGF-1 axis component is no more than three, or two components removed from GH, IGF-1, or the IGF-1 receptor. For example, an upstream component that is no more than two components removed may act through one or two intermediaries to modulate axis activity. In some embodiments, the GH/IGF-1 axis component is no more than one component removed (e.g., no more than one intermediary) between the component and GH, IGF-1, or the IGF-1 receptor. In another embodiment, the GH/IGF-1 axis component is no more than two components removed from PI(3) kinase, PTEN phosphatase, PI(3,4)P₂, and PI(3,4,5)P₃ phosphatidyl inositol kinases, AKT serine/threonine kinase (e.g., AKT-1, AKT-2, or AKT-3), or a Forkhead transcription factor. In one embodiment, the component is other than a heat shock protein. For example, a method can function without directly administering or directly modulating (activating or repressing a heat shock protein).

A “functional fragment” of a protein or nucleic acid refers to a polymer sequence (e.g., protein or nucleic acid sequence) of at least 5 subunits that is able to provide at least one activity (e.g., a biological activity) of the full-length protein or nucleic acid in vitro or in vivo. For example, functional fragments of a GH/IGF-1 axis component can be used to evaluate a test compound. Exemplary functional fragments of proteins are at least 40, 10, 50, 80, 100, 120, 150, or 200 amino acids in length. For example, the fragment may include an active or catalytic site, a regulatory site (e.g., a phosphorylation site or an allosteric regulator binding site), or a protein-protein interaction site. In one embodiment, with respect to cell surface components, the fragment is all or part of an extracellular or intracellular domain.

As used herein, “activity of the GH/IGF-1 axis” refers to the net effect of the axis components with respect to ability to stimulate GH secretion, increase IGF-1 levels, or increase IGF-1 receptor signalling. Accordingly, “downregulating the GH/IGF-1 axis” refers to modulating one or more components such that one or more of the following is reduced, e.g., decreased GH, decreased IGF-1, or decreased IGF-1 receptor signalling. For example, in some instances, GH levels are maintained but its action is inhibited; thus IGF-1 levels are decreased without decreasing GH levels. In some instances, both GH and IGF-1 levels are decreased.

A “direct antagonist” of a particular subject component includes (1) compounds that, at the protein level, directly bind or modify the subject component such that an activity of the subject component is decreased, e.g., by competitive or non-competitive inhibition, destabilization, destruction, clearance, or otherwise, and (2) compounds that, at the nucleic acid level, directly bind or modify a nucleic acid (e.g., chemically modify or alter the regulation thereof), e.g., a RNA or DNA, that encodes at least a region of the subject component or a complement thereof such that an activity of the subject component is decreased, e.g., by reducing transcription, or translation, or otherwise. Examples of antagonists at the protein level include antibodies, proteins, or peptides (e.g., fragments of naturally occurring ligands, random or semi-randomly generated binding peptides, and so forth), non-proteinaceous molecules, e.g., molecules less than 3000, 2000, 1000, 700, 500, 400, 300, or 200 in molecular weight or nucleic acid aptamers. Examples of antagonists at the nucleic acid level include RNAi, antisense RNAs, a ribozyme molecule, or molecule which bind to a regulatory element for the subject component (e.g., an artificial transcription factor, e.g., a zinc finger protein).

A “direct agonist” of a particular subject component includes (1) compounds that, at the protein level, directly bind or modify the subject component such that an activity of the subject component is increased, e.g., by activation, stabilization, altered distribution, or otherwise; (2) compounds that, at the protein level, themselves provide the activity of the subject component (e.g., a recombinant version of the subject component or an active fragment thereof); (3) compounds that, at the nucleic acid level, directly bind or modify a nucleic acid (e.g., chemically modify or alter the regulation thereof), e.g., a RNA or DNA, that encodes at least a region of the subject component or a complement. thereof such that an activity of the subject component is increased, e.g., by increasing transcription, translation, or otherwise; and (4) compounds that, at the nucleic acid level, themselves provide the activity of the subject component (e.g., a nucleic acid encoding a recombinant version of the subject component or an active fragment thereof, or a complement thereof). Examples of agonists at the protein level include activating antibodies, proteins, or peptides (e.g., fragments of naturally occurring ligands, random or semi-randomly generated binding peptides, and so forth), and non-proteinaceous molecules, e.g., molecules less than 3000, 2000, 1000, 700, 500, 400, 300, or 200 in molecular weight. Examples of agonists at the nucleic acid level include transgenes, coding nucleic acids (e.g., mRNAs or DNAs), or molecule which bind to a regulatory element for the subject component (e.g., an artificial transcription factor, e.g., a zinc finger protein).

Generally, a receptor exists in an active (Ra) and an inactive (Ri) conformation. Drugs that affect the receptor can alter the ratio of Ra to Ri (Ra/Ri). For example, a full agonist increases the ratio of Ra/Ri and can cause a “maximal”, saturating effect.

A partial agonist, when bound to the receptor, gives a response that is lower than that elicited by a full agonist. Thus, the Ra/Ri for a partial agonist is less than for a full agonist. However, the potency of a partial agonist may be greater or less than that of the full agonist.

An inverse agonist produces an effect opposite to that elicited by an agonist when it binds to the receptor. In this instance there is a shift in the equilibrium to Ri (e.g., an increase in Ri/Ra or a decrease in Ra/Ri). A super agonist causes an ultra-high response when bound to receptors, typically as a result of a particularly strong efficacy. Efficacy can be a function of the ligand's “on-rate” and “off-rate” for binding to the receptor.

Receptor desensitization is characterized by a loss in response to a ligand upon continued administration of a ligand. Desensitization can be mediated by a number of processes, e.g., agonist-induced receptor internalization and turnover, or diminished signal transduction within the cell beyond the cell surface receptor.

A subject with “normal” GH levels is one who would return a normal result using the glucose tolerance test in which glucose is ingested and blood levels of GH are measured by radioimmunoassay (RIA) or polyclonal immunoassay. A normal result for this test is characterized by less than 1 ng/mL of GH within 1 to 2 hours of an oral glucose load. However, GH levels of a subject with excessive GH, as in one with acromegaly may not decrease below 1 ng/mL after ingesting glucose. Because GH levels oscillate every twenty to thirty minutes and varies in level according to the time of day, stress level, exercise, etc., a standard means of determining if GH levels are excessive is to administer glucose. This approach normalizes GH and is less affected by the pulsatility of GH, age, gender, or other factors. Alternatively or as a confirmation, since IGF-1 levels are invariably increased in acromegalic individuals, IGF-1 levels can be measured and compared to age and gender matched normal controls.

The term “an indicator of GH/IGF-1 axis activity” refers to a detectable property of the GH/IGF-1 axis that is indicative of activity of the axis. Exemplary properties include circulating GH concentration, circulating IGF-1 concentration, frequency of GH pulses, amplitude of GH pulses, GH concentration in response to glucose, IGF-1 receptor phosphorylation, and IGF-1 receptor substrate phosphorylation.

A “test compound” or “candidate compound” is any chemical compound, which may or may not affect the GH/IGF-1 axis. Exemplary test compounds include candidate proteins, peptides, peptidomimetics, peptoids, small molecules or other drugs. Exemplary small molecules have a molecular weight of less than 7000, 5000, 4000, 3000, 2000, 1000, 700, 500, 400, 300, or 200 Daltons. Small molecules include, for example, benzolactams and spiroindanylpiperadines. A test compound can be soluble or insoluble in an aqueous solution. In one embodiment, an exemplary test compound is an agonist or antagonist of a compound described herein, e.g., a somatostatin agonist.

An “age-associated disorder” or “age-related disorder” is a disease or disorder whose incidence is at least 1.5 fold higher among human individuals greater than 60 years of age relative to human individuals between the ages of 30-40, at the time of filing of this application and in a selected population of greater than 100,000 individuals. A preferred population is a United States population. A population can be restricted by gender and/or ethnicity.

A “geriatric disorder” is a disease or disorder whose incidence, at the time of filing of this application and in a selected population of greater than 100,000 individuals, is at least 70% among human individuals that are greater than 70 years of age. In one embodiment, the geriatric disorder is a disorder other than cancer or a cardio-pulmonary disorder. A preferred population is a United States population. A population can be restricted by gender and/or ethnicity.

A disorder having an “age-associated susceptibility factor” refers to a disease or disorder whose causation is mediated by an externality, but whose severity or symptoms are substantially increased in human individuals over the age of 60 relative to human individuals between the ages of 30-40, at the time of filing of this application and in the United States population. For example, pneumonia is caused by pathogens, but the severity of the disease is greater in humans over the age of 60 relative to human individuals between the ages of 30-40.

A “neoplastic disorder” is a disease or disorder characterized by cells that have the capacity for autonomous growth or replication, e.g., an abnormal state or condition characterized by proliferative cell growth. An “age-associated neoplastic disorder” is a neoplastic disorder that is also an age-associated disorder.

A “non-neoplastic disorder” is a disease or disorder that is not characterized by cells that have the capacity for autonomous growth or replication. An “age-associated non-neoplastic disorder” is a non-neoplastic disorder that is also an age-associated disorder.

A “neurological disorder” is a disease or disorder characterized by an abnormality or malfunction of neuronal cells or neuronal support cells (e.g., glia or muscle). The disease or disorder can affect the central and/or peripheral nervous system. Exemplary neurological disorders include neuropathies, skeletal muscle atrophy, and neurodegenerative diseases, e.g., a neurodegenerative disease other than one caused at least in part by polyglutamine aggregation. Exemplary neurodegenerative diseases include: Alzheimer's, Amyotrophic Lateral Sclerosis (ALS), and Parkinson's disease. An “age-associated neurological disorder is a neurological disorder that is also an age-associated disorder.

A “cardiovascular disorder” is a disease or disorder characterized by an abnormality or malfunction of the cardiovascular system, e.g., heart, lung, or blood vessels. Exemplary cardiovascular disorders include: cardiac dysrhythmias, chronic congestive heart failure, ischemic stroke, coronary artery disease and cardiomyopathy. An “age-associated cardiovascular disorder is a cardiovascular disorder that is also an age-associated disorder.

A “metabolic disorder” is a disease or disorder characterized by an abnormality or malfunction of metabolism. One category of metabolic disorders are disorders of glucose or insulin metabolism An “age-associated metabolic disorder is a metabolic disorder that is also an age-associated disorder.

A “dermatological disorder” is a disease or disorder characterized by an abnormality or malfunction of the skin. A “dermatological tissue condition” refers to the skin and any underlying tissue (e.g., support tissue) which contributes to the skins function and/or appearance, e.g., cosmetic appearance.

Exemplary diseases and disorders that are relevant to certain implementations include: cancer (e.g., breast cancer, colorectal cancer, CCL, CML, prostate cancer); skeletal muscle atrophy; adult-onset diabetes; diabetic nephropathy, neuropathy (e.g., sensory neuropathy, autonomic neuropathy, motor neuropathy, retinopathy); obesity; bone resorption; age-related macular degeneration, ALS, Alzheimer's, Bell's Palsy, atherosclerosis, cardiovascular disorders (e.g., cardiac dysrhythmias, chronic congestive heart failure, ischemic stroke, coronary artery disease and cardiomyopathy), chronic renal failure, type 2 diabetes, ulceration, cataract, presbiopia, glomerulonephritis, Guillan-Barre syndrome, hemorrhagic stroke, short-term and long-term memory loss, rheumatoid arthritis, inflammatory bowel disease, multiple sclerosis, SLE, Crohn's disease, osteoarthritis, Parkinson's disease, pneumonia, and urinary incontinence. In addition, many neurodegenerative disorders and disorders associated with protein aggregation (e.g., other than polyglutamine aggregation) or protein misfolding can also be age-related. Symptoms and diagnosis of diseases are well known to medical practitioners. The compositions may also be administered to individuals being treated by other means for such diseases, for example, individuals being treated with a chemotherapeutic (e.g., and having neutropenia, atrophy, cachexia, nephropathy, neuropathy) or an elective surgery.

Abbreviations:

GH=Growth Hormone

GH-R=Growth Hormone Receptor

IGF=Insulin-like Growth Factor

GHRH=GH Releasing Hormone

GHRH-R=GH Releasing Hormone Receptor

GHS=GH Secretagogue

GHS-R=GH Secretagogue Receptor

SST=Somatostatin

SST-R=Somatostatin Receptor

PI=phosphoinositol

GADD=Growth Arrest DNA Damage Response

The details of one or more embodiments of the invention are set forth in the description below. Other features, objects, and advantages of the invention will be apparent from the description and the claims. The contents of all references, pending patent applications and published patents, cited throughout this application are hereby expressly incorporated by reference.

### BRIEF DESCRIPTION OF THE DRAWINGS

FIG. 1 is a schematic of the GH/IGF-1 axis.

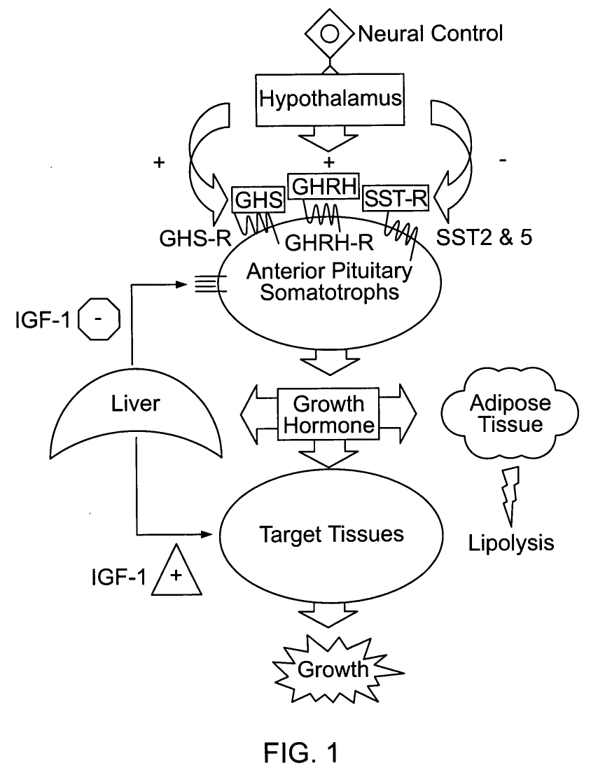

FIG. 2 is a schematic of some insulin/IGF-1 signalling components.

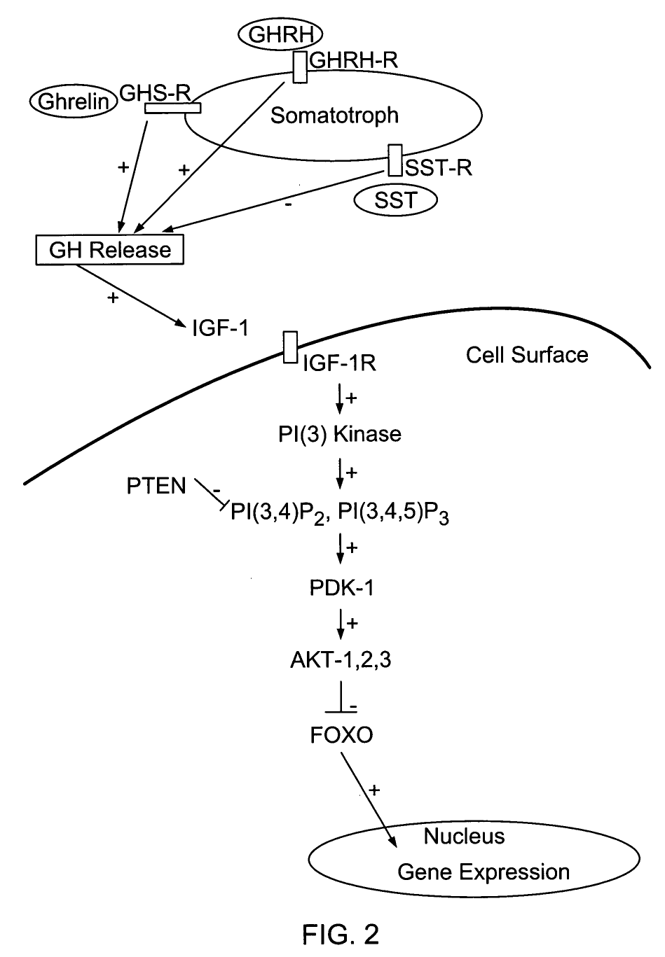

FIG. 3 is a schematic of a modification of L-054,522, an agonist, to provide an antagonist.

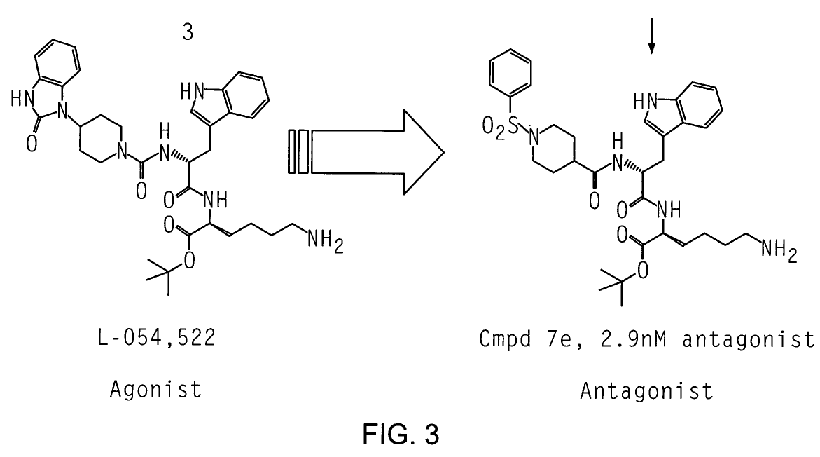

### DETAILED DESCRIPTION

This disclosure provides, inter alia, treatments and compositions that alter life span regulation and cellular responses to diseases and disorders by antagonizing the GH/IGF-1 axis. Also provided are methods of screening for agents that can modulate the GH/IGF-1 axis.

Although naturally the activity of the GH/IGF-1 axis declines in older human individuals, a further reduction from the norm for a particular adult age is beneficial. In particular, the reduction of GH/IGF-1 axis activity can alter lifespan regulation within a subject and beneficially affects a number of age-related disorders.

The net effect of downregulation can be manifested, e.g., as reduced GH levels, reduced IGF-1 levels, reduced IGF-1 receptor signaling, reduced GHRH levels, reduced GHS levels, or increased somatostatin levels. In many cases, it is useful to reduce such levels below the norm, but to retain at least a detectable amount, e.g., a non-zero level. For example, partial reduction can include reducing a level to a resulting level that is less than 90, 80, 70, 60, 50, or 30% and/or greater 70, 65, 60, 55, 50, 45, 40, or 15% of the initial level of a subject. In another example, partial reduction can include reducing a level to a resulting level that is less than 90, 80, 70, 60, 50, or 30% and/or greater than 70, 65, 60, 55, 50, 45, 40, or 15% of the average level among normal individuals having the same age and gender as the subject. Typically, the subject is an adult (e.g.,.a human adult having an age of at least 18, 21, 24, or 28 years) without defects in the GH/IGF-1 axis, and thus does not have acromegaly. Acromegaly is a disorder of excessive GH production which stimulates excessive IGF-1 production. A glucose tolerance test can be used determine if a person has excessive GH. GH in a normal person decreases to less than 1 or 2 ng/mL after ingestion of sugar whereas in the acromegalic person, GH does not decrease below 1 or 2 ng/mL.

In one embodiment, the subject has a disorder that is caused at least in part by protein misfolding or protein aggregation (e.g., other than as a result of polyglutamine aggregation), or it has a neurodegenerative disorder. Disorders involving a misfolded protein have been identified in mammals. These disorders include, for example, Parkinson's disease; prion diseases (including Creutzfeldt-Jakob disease (CJD), Fatal Familia insomnia (FFI), Gerstmann-Straussler-Scheinker disease (GSS), mad cow disease, Scrapie, and kuru); Familial Amyloid Polyneuropathy, Tauopathies (including Pick Disease, Lobar Atrophy, and Frontotemporal dementia); polyglutamine aggregation disorders, Fragile-X syndrome, myotonic dystrophy, Haw River Syndrome, hereditary ataxias, and Machado Joseph disease. Alzheimer's disease is an example of a disease in which amyloid is produced, e.g., as a result of protein aggregation. Amyloid is also produced in other disorders, e.g., due to transthyretin aggregation etc.

Moreover, aberrant aggregation is a common feature of many neurodegenerative diseases, not only disorders caused at least in part by polyglutamine aggregation. For example, aberrant aggregation is also a principal factor in Alzheimer's disease (amyloid plaques) and Parkinson's disease (Lewy bodies). The formation of protein aggregates may be involved at some stage in disease pathogenesis in a variety of disorders, neurological and otherwise (see, e.g., diseases caused by protein misfolding). There are a variety of methods that can be used to down regulate the GH/IGF-1 axis. For example, axis activity can be reduced by targeting a particular component of the axis. Depending on the component's function in the axis, it may be appropriate to inhibit its activity or to promote its activity. For example, axis activity can be reduced by agonizing an inhibitory component of the axis or antagonizing a component that promotes or is required for axis activity. Exemplary targets and the desired activity used against these targets to reduce axis activity are listed in Table 1.

TABLE 1
Exemplary Axis Components

| Target | Desired Activity |
|---|---|
| SST2 & SST5 | Agonist |
| GHRH/GHRH-R | Antagonist |
| GHS/GHS-R | Antagonist |
| GH/GH-R | Antagonist |
| IGF-1/IGF-1R | Antagonist |
| PI(3) kinase | Inhibitor |
| PTEN | Agonist |
| PDK-1 | Inhibitor |
| Akt-1, -2, -3 | Inhibitor |
| Forkhead | Agonist |

Of course, some molecules may fit more than one of the above classifications.

As seen in Table 1, these molecules include molecules which can target extracellular molecules: for example,: a GH antagonist; a GH receptor antagonist; a GHRH antagonist; a GHRH receptor antagonist; IGF-1 receptor antagonist; IGF-1 antagonist; a somatostatin agonist; and; a somatostatin receptor agonist; as well as molecule that target intracellular molecules.

Somatostatin Agonists

Somatostatin and somatostatin agonists can be used to downregulate the GH/IGF-1 axis. As used herein a “somatostatin agonist” is a compound that has at least one biological function of somatostatin and that can alter regulation of the GH/IGF-1 axis. The recombinant form of somatostatin as well as somatostatin octapeptides have been used to treat acromegaly. One useful somatostatin agonist is L-054,522. See, e.g., Pasternak et al. (1999) Bioorganic & Medicinal Chemistry Letters which also provides L-054,522 related compounds with improved bioavailability; and Yang et al. (1998) Proc Nat Acad USA 95:10836. L-054,522 binds to human SST2 with an apparent K_{d} of 0.01 nM and is highly selective. One exemplary L-054,522 compound has the following structure:

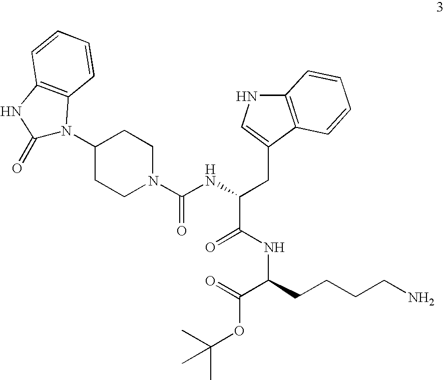

Other useful somatostatin agonists include BIM-23244, BIM-23197, BIM-23268, octreotide, TT-232, butreotide, lanreotide, and vapreotide. Octreotide and lanreotide are currently approved for treatment of acromegaly. These bind the receptors on the anterior pituitary gland and function to lower the production and secretion of GH.

Somatostatin is a hypothalamic factor that, among other biological functions, suppresses the secretion of GH from the anterior pituitary. It is produced by a large number of tissues. Due to its rapid degradation and clearance, somatostatin is not a truly circulating hormone. It is produced locally to its site of function, presumably to prevent inappropriate activation of receptors in tissues throughout the body. In developing drugs that mimic somatostatin, a key goal is to increase its stability thus extending its circulating half-life. In one embodiment, a somatostatin analog has local tissue specificity. For example, it may bind a subset of the five distinct receptor subtypes that bind to somatostatin, particularly the SST2 or SST5 receptors.

EPO1492 (Cortistatin 8), is a somatostatin antagonist which has been shown to inhibit feeding in animal studies. EPO1492 is an 8 amino acid peptide somatostatin analogue. See generally, e.g., WO 03/004518 and WO 02/08250.

GH Antagonists

GH antagonists include molecules which antagonize production (e.g., synthesis or secretion) of GH. GH antagonists include a naturally occurring antagonist—somatostatin—and pharmaceuticals. Exemplary pharmaceuticals include those used to treat acromegaly (a disorder of excessive GH) by antagonizing GH are the somatostatin agonists (see above) and dopamine agonists (bromocriptine (Parlodel), pergolide (Permax), and cabergoline (Dostinex)).

Dopamine Agonists. The dopamine agonists, bromocriptine, pergolide, and cabergoline also decrease GH production. Thus, these agents can be used to alter life span regulation. Bromocriptine, pergolide, and cabergoline are synthetic compounds that act like the naturally occurring compound dopamine to reduce GH secretion.

GH Receptor Antagonists

GH receptor antagonists include molecules that antagonize the function of the GH receptor, for example, by preventing binding of GH or GH receptor dimerization.

Pegvisomant. An example of a GH receptor antagonist is Pegvisomant. Pegvisomant (Somavert) is a modified human GH in which nine amino acids have been replaced thus preventing receptor dimerization. Normally a single GH molecule binds to two GH receptor molecules to allow their dimerization. These amino acid changes at the dual receptor binding site of human GH allow Pegvisomant to bind more strongly to a single receptor molecule with inhibition of binding to the second receptor molecule, thus preventing dimerization of the GH receptor. Polyethlyene glycol polymers on Pegvisomant decrease its rate of clearance, reduce its immunogenicity, and enhance its bioactivity. Pegvisomant is one available treatment for acromegaly. It has been observed that Pegvisomant administered subcutaneously causes a dose dependent reduction in IGF-1 levels.

GHS/GHS-R Antagonists

Antagonists of growth hormone secretagogues (GHS) and GHS receptors can be used to downregulate the GH/IGF-1 axis and thereby modulate lifespan regulation and/or aging. An endogenous ligand (i.e., an endogenous GHS) for the GHS receptor is ghrelin.

An exemplary GHS antagonist is [D-Lys3]-GHRP-6, antagonist for Growth Hormone Releasing Peptide 6 (see also His-D-Trp-D-Lys-Trp-D-Phe-Lys-NH2; Sigma-Aldrich Product No. G4535). Other antagonists include compounds that interact with the GHS-receptor. For example, antibodies to ghrelin can be used as antagonists. See, e.g., Nakazato et al. (2001) Nature 409:194. Similarly, ligands that bind to GHS receptors, e.g., antibody ligands, can be used to antagonize the axis.

GHRH Antagonists

GHRH is a peptide present in the hypothalamus which causes GH release from the anterior pituitary by interacting with specific GHRH receptors. A “GHRH antagonist” antagonizes the function of GHRH, e.g., by preventing or competing for receptor binding. GHRH antagonists decrease secretion of GH by the anterior pituitary somatotroph. An example of a GHRH antagonist is [N-acetyl-Tyr¹,D-Arg²] GHRH¹⁻²⁹NH₂, herein referred to as the “standard GHRH antagonist.” The standard GHRH antagonist, which is a modified version of the first 29 residues of GHRH (the shortest fragment of GHRH that possesses GH-releasing capability and binding properties) lowers spontaneous GH secretion and inhibits human GH secretory response to exogenous GHRH (Nargund et al., Journal of Medicinal Chemistry 41:3103-3127, 1998; Dimaraki et al., Proceedings of the 83^{rd} Meeting of the Endocrine Society, p. 97, Abstract OR22-3). The sequence of the first 29 residues of GHRH that still possesses GH-releasing capability and binding properties, thus referred to as the bioactive core of GHRH, is as follows:

Tyr¹-Ala-Asp-Ala-Ile-Phe-Thr-Ans-Ser-Tyr-Arg-Lys-Val-Leu-Gly-Gln-Leu-Ser-Ala-Arg-Lys-Leu-Leu-Gln-Asp-Ile-Met-Ser-Arg²⁹

The standard GHRH antagonist and many other antagonists have a D-Arg in the second position which confers its antagonist activity. More potent GHRH inhibitors can be constructed with certain hydrophobic and helix-stabilizing amino acid substitutions, such as para-chlorophenylalanine (Phe(4-Cl)) in position six, α-aminobutyric acid (Abu) in position fifteen, and norleucine (Nle) in position 27, combined with a hydrophobic N-terminal acyl moiety, such as iso-butyryl (Ibu-), phenylacetyl (PhAc-) or 1-naphthylacetyl (Nac-) (Schally and Varga, Trends Endocrinol. Metab., 10:382-391, 1999; Zarandi et al., Proc. Natl. Acad. Sci. USA 91:12298-12302, 1994; U.S. Pat. No. 6,057,422). Replacement of the Arg residue in position 29 with agmatine (Agm), combined with the N-terminal acylation of the analogs contributes to enzymatic stability and protracted antagonistic activity in vivo as compared to the standard GHRH antagonist. Small molecule mimetics of these antagonists can also be produced based on the 3-dimensional structures described above. In addition to inhibiting GH release, GHRH antagonists may indirectly decrease pituitary production of GH and of GH-mediated hepatic synthesis of IGF-1.

Accordingly, a GHRH antagonist can be used to attenuate activity of the GH/IGF-1 axis.

Agonist-Based Screening

In one aspect, the invention features a method of identifying an antagonist of the GH/IGF-1 axis. The method is based upon information about agonists of the axis. In this method, the agonist serves as a starting point for a screen to identify chemically and structurally related compounds that may inhibit the axis, in particular compounds that antagonize rather than agonize the axis.

The method takes advantage of the fact that the agonist interacts with a component of the axis. Modification or similarity to the agonist may retain some physical aspects of the interaction with this component but may provide new properties that result in the opposite functional effect. For example, it is known that a dimeric ligand that agonizes a cell surface receptor, may antagonize it in monomeric form. Although the monomer may bind more poorly than the dimer, modification of the monomer to generate an additional binding interface may produce an effective antagonist.

A variety of processes can be used to implement the above method. These processes can be used in conjunction with a screening method described herein.

Chemical Libraries. In one example, combinatorial chemical libraries can be produced that sample chemical compounds that are structurally or chemically related. For example, a scaffold is selected based on information about the known agonist. Then various positions on the scaffold are modified in combination to produce a large number of different compounds. The diversity of particular positions can be precisely controlled.

Methods for producing chemical libraries are well known. See, for example, Cox et al.. (2000) Prog Med Chem 37:83; Sternson (2001) Org Lett 3(26):4239-42; Tam et al.. (1998) J. Am Chem. Soc. 120:8565; 1: Floyd et al.. (1999) Prog Med Chem. 36:91-168.; Rohrer et al.. (1998) Science.;282(5389):737-40; Komarov et al. (1999) Science. 285(5434):1733-7; Mayer et al.. (1999) Science. 286(5441):971-4.

Members of a chemical library can be tagged. In such libraries, the identity and composition of each member of the library is uniquely specified by the label or “tag” which is physically associated with it and hence the compositions of those members that bind to a given target or that have a particular activity are specified directly (see, e.g., Ohlmeyer et al., 1993, Proc. Natl. Acad. Sci. USA 90:10922-10926; Brenner et al., 1992, Proc. Natl. Acad. Sci. USA 89:5381-5383; Lerner et al., PCT Publication No. WO 93/20242). In other examples of such libraries, the library members are created by step wise synthesis protocols accompanied by complex record keeping, complex mixtures are screened, and deconvolution methods are used to elucidate which individual members were in the sets that had activity (e.g., binding or biological activity), and hence which synthesis steps produced the members and the composition of individual members (see, e.g., Erb et al., 1994, Proc. Natl. Acad. Sci. USA 91:11422-11426).

Structure-Activity Relationships and Structure-Based Design. It is also possible to use structure-activity relationships (SAR) and structure-based design principles to produce an agonists from an antagonist. SARs provide information about the activity of related compounds in at least one relevant assay. Correlations are made between structural features of a compound of interest and an activity. For example, it may be possible by evaluating SARs for a family of compounds related to a GH/IGF-1 axis agonist to identify one or more structural features required for the agonist's activity. A library of compounds can then be produced that vary these features. In a related example, features required for agonist activity, but not for binding to the component of the axis that is the target of the agonist can be varied.

Structure-based design can include determining a structural model of the physical interaction of a GH/IGF-1 axis agonist and its target. The structural model can indicate how an antagonist of the target can be engineered.

Both the SAR and the structure-based design approach can be used to identify a pharmacophore. Pharmacophores are a highly valuable and useful concept in drug discovery and drug-lead optimization. A pharmacophore is defined as a distinct three dimensional (3D) arrangement of chemical groups essential for biological activity. Since a pharmaceutically active molecule must interact with one or more molecular structures within the body of the subject in order to be effective, and the desired functional properties of the molecule are derived from these interactions, each active compound must contain a distinct arrangement of chemical groups which enable this interaction to occur. The chemical groups, commonly termed descriptor centers, can be represented by (a) an atom or group of atoms; (b) pseudo-atoms, for example a center of a ring, or the center of mass of a molecule; (c) vectors, for example atomic pairs, electron lone pair directions, or the normal to a plane. Once formulated a pharmacophore can be used to search a database of chemical compound, e.g., for those having a structure compatible with the pharmacophore. See, for example, U.S. Pat. No. 6,343,257 ; Y. C. Martin, 3D Database Searching in Drug Design, J. Med. Chem. 35, 2145(1992); and A. C. Good and J. S. Mason, Three Dimensional Structure Database Searches, Reviews in Comp. Chem. 7, 67(1996). Database search queries are based not only on chemical property information but also on precise geometric information. Computer-based approaches can use database searching to find matching templates; Y. C. Martin, Database searching in drug design, J. Medicinal Chemistry, vol. 35, pp 2145-54 (1992), which is herein incorporated by reference. Existing methods for searching 2-D and 3-D databases of compounds are applicable. Lederle of American Cyanamid (Pearl River, N.Y.) has pioneered molecular shape-searching, 3D searching and trend-vectors of databases. Commercial vendors and other research groups also provide searching capabilities (MACSS-3D, Molecular Design Ltd. (San Leandro, Calif.); CAVEAT, Lauri, G. et al., University of California (Berkeley, Calif.); CHEM-X, Chemical Design, Inc. (Mahwah, N.J.)). Software for these searches can be used to analyze databases of potential drug compounds indexed by their significant chemical and geometric structure (e.g., the Standard Drugs File (Derwent Publications Ltd., London, England), the Bielstein database (Bielstein Information, Frankfurt, Germany or Chicago), and the Chemical Registry database (CAS, Columbus, Ohio)).

Once a compound is identified that matches the pharmocophore, it can be tested for activity, e.g., for binding to a component of the GH/IGF-1 axis and/or for a biological activity, e.g., modulation of the axis, e.g., downregulation of the axis. See, e.g., “Screening Methods” below.

The following are examples of known agonists of the GH/IGF-1 axis. Each of these agonists can serve as a base compound for identifying an antagonist of the axis.

GHRH Agonists

Agonists of GHRH serve to increase GH. Examples of known GHRH agonists are GHRH¹⁻⁴⁴NH₂ and GHRH¹⁻²⁹NH₂. One type of structural library that can be based on these agonists are peptide libraries in which subregions of the 29 amino acid peptide sequence are varied, and tested for modulation of the IGF-1 axis.

GHS Agonists

Another class of molecules that can be modified to find an agent that down regulates the pathway is the class of GHS agonists. At least some of these agonists have been used to treat patients with a GH deficiency. Exemplary GHS agonists include, ghrelin, GHS-6, MK-0677 (L-163,191) and L-739,943. The endogenous GH secretagogue is ghrelin. MK-0677 is a spiroindanylpiperadine with potent GH-releasing effects when administered orally and parenterally (Patchett (1995) Proc Nat Acad USA 92:7001). Its structure is as follows:

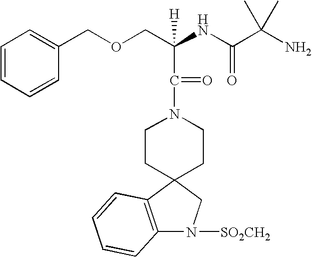

L-739,943, a potent, orally bioavailable benzolactam GH secretagogue, is obtained from zwitterionic L-692,429 through modification of its amino acid side chain and replacement of the acidic 2′-tetrazole with the neutral and potency enhancing 2′-(N-methylaminocarbonylamino)methyl substituent. (De Vita et al., J Med Chem 41:1716-28, 1998). Other GH agonists include penta-, hex-, and heptapeptide analogs that specifically stimulated GH secretion from the anterior pituitary gland in a dose-dependent manner in vitro and in vivo. These include Leu- and Met-Enkephalin, GHRP-1 to GHRP-6, and hexarelin (Root and Root (2002), supra).

Various libraries of compounds can be designed based on these compounds and screened to identify compounds that downregulate the GH/IGF-1 axis.

Other general agonists of GH action can also be used as a basis for identifying an axis antagonist. For example, arginine is a potent cholinergic agonist that has been successful in stimulating GH secretion even in the elderly in whom many GH agonists have not been as successful. Arginine analogs and pro-drugs can also be used as starting points for identifying an antagonist of the pathway.

Still other agonists include SM-130686, an oxindole derivative (available, e.g., from Sumitomo), NN703 and hexarelin. Similarly, it is possible to use selective antagonists of somatostatin receptors, e.g., SST2, to develop somatostatin receptor agonists. Exemplary somatostatin receptor antagonists include BIM-23454 and BIM-23627 (Biomeasure). EP-01572 can be used to develop GHS-R antagonists. Still other agonists that can be modified include EP-80317.

The somatostatin receptor 2 (SSTR2) and GHS (ghrelin) receptor are G-protein coupled receptors that control GH release at the level of the hypothalamic-pituitary axis. Whereas an agonist of SSTR2 blocks GH release in vitro and in vivo, an antagonist of the GHS-R is expected to produce the same result There are structural similarities between some known agonists of SSTR2 and agonists for GHS-R, namely those of the spiropiperdine class. Some SSTR2-active compounds also possess agonist activity at GHS-R as assessed by a second messenger-signal transduction assay. Through medicinal chemistry approaches, molecules can be designed that modulate both SSTR2 and GHS receptors. Such molecules are referred to as dual activity ligands. A dual activity ligand may possess SSTR2 agonist activity concurrent with GHS receptor antagonist activity, to yield an additive or synergistic inhibition of GH release. Operating through these receptors dual activity ligands provide an additional means to decrease signaling through the GH/IGF-1 axis, ultimately stimulating forkhead transcriptional activity.

Artificial Transcription Factors

Artificial transcription factors can also be used to regulate genes that are regulated by Forkhead. For example, an artificial transcription factor that has the binding specificity of Forkhead can be used to substitute for or augment Forkhead function. For example, the artificial transcription factor can be engineered to bind to the nucleic acid sequence: TTGTTTAC or any other sequence specifically bound by Forkhead. The protein can be designed or selected from a library. For example, the protein can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Nat. Acad. Sci. USA 92:344 for, among other things, methods for creating libraries of varied zinc finger domains.

Optionally, the zinc finger protein can be fused to a transcriptional regulatory domain, e.g., an activation domain to activate transcription or a repression domain to repress transcription. The zinc finger protein can itself be encoded by a heterologous nucleic acid that is delivered to a cell or the protein itself can be delivered to a cell (see, e.g., U.S. Pat. No. 6,534,261. The heterologous nucleic acid that includes a sequence encoding the zinc finger protein can be operably linked to an inducible promoter, e.g., to enable fine control of the level of the zinc finger protein in the cell.

Screening Assays

A test compound can be evaluated for its effect on the GH/IGF-1 axis or for its ability to interact with a GH/IGF-1 component. Methods include in vitro and in vivo assays. Interactions include, for example, binding a target component, altering a covalent bond in a target component, or altering a biological or physiological function of a target compound (e.g., altering production, stability, or degradation of a target component). A test compound that modulates the GH/IGF-1 axis (e.g., downregulates the axis) can be prepared as a pharmaceutical composition (see below) and administered to a subject.

The test compounds can be obtained, for example, as described above (e.g., based on information about an agonist) or using any of the numerous combinatorial library method. Some exemplary libraries include: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. These approaches can be used, for example, to produce peptide, non-peptide oligomer or small molecule libraries of compounds (see, e.g., Lam (1997) Anticancer Drug Des. 12:145).

A biological library includes polymers that can be encoded by nucleic acid. Such encoded polymers include polypeptides and functional nucleic acids (such as nucleic acid aptamers (DNA, RNA), double stranded RNAs (e.g., RNAi), ribozymes, and so forth). The biological libraries and non-biological libraries can be used to generate peptide libraries. Another example of a biological library is a library of dsRNAs (e.g., siRNAs), or precursors thereof. A library of nucleic acids that can be processed or transcribed to produce double-stranded RNAs (e.g., siRNAs)) is also featured.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.). In many cases, a high throughput screening approach to a library of test compounds includes one or more assays, e.g., a combination of assays. Information from each assay can be stored in a database, e.g., to identify candidate compounds that can serve as leads for optimized or improved compounds, and to identify SARs.

Cell-Based Assays. In one embodiment, a cell-based assay is used to evaluate a test compound. The cell, for example, can be of mammalian origin, (e.g., from a human, a mouse, rat, primate, or other non-human), or of non-mammalian origin (e.g., Xenopus, zebrafish, or an invertebrate such as a fly or nematode). In some cases, the cell can be obtained from a transgenic organism, e.g., an organism which includes a heterologous GH/IGF-1 axis component, (e.g., from a mammal, e.g., a human).

In one example, a cell which expresses a GH/IGF-1 axis protein or polypeptide or biologically active portion thereof is contacted with a test compound, and the ability of the test compound to modulate the GH/IGF-1 axis is determined. Determining the ability of the test compound to modulate the GH/IGF-1 axis can be accomplished by monitoring, for example, GH and/or IGF-1 levels, e.g., by radioimmunoassay. For example, the assay can include evaluate GH or IGF-1 synthesis and release.

See Example 1, below which describes an assay using cultured pituitary cells. It is also possible to monitor an intracellular component of the GH/IGF-1 axis, e.g., abundance, activity or post-translational modification state of a PI(3)Kinase, a phosphatase (e.g., PTEN), a phosphoinositol kinase; or a serine-threonine kinase (e.g., an AKT kinase). Changes in post-translational modification can be monitored using modification specific antibodies, changes in electrophoretic mobility, and mass spectroscopy, for example.

Another exemplary cellular assay includes contacting a hormone responsive cell with a hormone (e.g., somatostatin, GH or IGF-1) in the presence of the test compound and evaluating a parameter (e.g., a qualitative or quantitative property) of the cell (e.g., expression of one or a profile of genes, abundance of one or more proteins, and so forth). Alteration of the parameter relative to a control cell or a reference parameter (e.g., a reference value) indicates that the test compound can modulate the responsiveness of the cell.

Still other cell-based assays including contacting cells with the test compound and evaluating resistance to a stress, for example, hypoxia, DNA damage (genotoxic stress), or oxidative stress. For example, it is possible to determine whether hypoxia-mediated cell death is attenuated by the test compound.

Cell-Free Assays. In addition to cell-based assays, cell-free assays can also be used. In one example, the ability of the test compound to modulate interaction between a first GH/IGF-1 axis component and a second axis component is evaluated, e.g., interaction between GH and the GH receptor or GHRH and the GHRH receptor. This type of assay can be accomplished, for example, by coupling one of the components, with a radioisotope or enzymatic label such that binding of the labeled component to the other GH/IGF-1 axis component can be determined by detecting the labeled compound in a complex. A GH/IGF-1 axis component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product.

Competition assays can also be used to evaluate a physical interaction between a test compound and a target. For example, Pong et al.. (1996) Mol Endocrinol 10:57 describes an assay which detects the displacement of a radiolabeled MK-0677 molecule from pituitary membranes.

In yet another embodiment, a cell-free assay is provided in which a GH/IGF-1 axis protein or biologically active portion thereof is contacted with a test compound and the ability of the test compound to bind to the GH/IGF-1 axis protein or biologically active portion thereof is evaluated. Preferred biologically active portions of the GH/IGF-1 axis proteins to be used in assays of the present invention include fragments which participate in interactions with non-GH/IGF-1 axis molecules, e.g., an ectodomain of a cell surface receptor, a cytoplasmic domain of a cell surface receptor, a kinase domain, and so forth.

Soluble and/or membrane-bound forms of isolated proteins (e.g., GH/IGF-1 axis components and their receptors or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate. In another example, the axis component can reside in a membrane, e.g., a liposome or other vesicle.

Cell-free assays involve preparing a reaction mixture of the target protein (e.g., the GH/IGF-1 axis component) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; Seethala et al.. (1998) Anal Biochem. 255:257. Fluorescence polarization can be monitored in multiwell plates, e.g., using the Tecan Polarion™ reader. See, e.g., Parker et al. (2000) Journal of Biomolecular Screening 5:77-88; and Shoeman, et al. (1999) 38, 16802-16809.

In another embodiment, determining the ability of the GH/IGF-1 axis component protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the axis component is anchored onto a solid phase. The axis component/test compound complexes anchored on the solid phase can be detected at the end of the reaction, e.g., the binding reaction. For example, the axis component can be anchored onto a solid surface, and the test compound, (which is not anchored), can be labeled, either directly or indirectly, with detectable labels discussed herein.

It may be desirable to immobilize either the GH/IGF-1 axis component or an anti-GH/IGF-1 axis component antibody to facilitate separation of complexed from uncomplexed forms of one or both of the proteins, as well as to accommodate automation of the assay. Binding of a test compound to a GH/IGF-1 axis component protein, or interaction of a GH/IGF-1 axis component protein with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/GH/IGF-1 axis component fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or GH/IGF-1 axis component protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of GH/IGF-1 axis component binding or activity determined using standard techniques.

Other techniques for immobilizing either a GH/IGF-1 axis component protein or a target molecule on matrices include using conjugation of biotin and streptavidin. Biotinylated GH/IGF-1 axis component protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a GH/IGF-1 axis component protein or target molecules but which do not interfere with binding of the GH/IGF-1 axis component protein to its target molecule. Such antibodies can be derivatized to the wells of the plate, and unbound target or the GH/IGF-1 axis component protein trapped in the wells by antibody conjugation. Methods for detecting such complexes, in addition to those described above for the GST-immobilized complexes, include immunodetection of complexes using antibodies reactive with the GH/IGF-1 axis component protein or target molecule, as well as enzyme-linked assays which rely on detecting an enzymatic activity associated with the GH/IGF-1 axis component protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11: 141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the GH/IGF-1 axis component protein or biologically active portion thereof with a known compound which binds a GH/IGF-1 axis component to form an assay mixture, contacting the assay mixture with a test compound, and determining the ability of the test compound to interact with a GH/IGF-1 axis component protein, wherein determining the ability of the test compound to interact with the GH/IGF-1 axis component protein includes determining the ability of the test compound to preferentially bind to the GH/IGF-1 axis component or biologically active portion thereof, or to modulate the activity of a target molecule, as compared to the known compound.

The target products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target product. Such compounds can include, but are not limited to molecules such as antibodies, peptides, and small molecules. The preferred targets/products for use in this embodiment are the GH/IGF-1 axis components. In an alternative embodiment, the invention provides methods for determining the ability of the test compound to modulate the activity of a GH/IGF-1 axis component protein through modulation of the activity of a downstream effector of a GH/IGF-1 axis component target molecule. For example, the activity of the effector molecule on an appropriate target can be determined, or the binding of the effector to an appropriate target can be determined, as previously described.

To identify compounds that interfere with the interaction between the target product and its cellular or extracellular binding partner(s), a reaction mixture containing the target product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target product can also be compared to complex formation within reaction mixtures containing the test compound and mutant target product. This comparison can be important in those cases wherein it is desirable to identify compounds that disrupt interactions of mutant but not normal target products.

These assays can be conducted in a heterogeneous or homogeneous format. Heterogeneous assays involve anchoring either the target product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target product and the interactive cellular or extracellular binding partner product is prepared in that either the target products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target product-binding partner interaction can be identified.

In yet another aspect, the GH/IGF-1 axis component proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with the GH/IGF-1 axis component (“GH/IGF-1 axis component-binding proteins” or “GH/IGF-1 axis component-bp”) and are involved in GH/IGF-1 axis component activity. Such GH/IGF-1 axis component-bps can be activators or inhibitors of signals by the GH/IGF-1 axis component proteins or GH/IGF-1 axis component targets as, for example, downstream elements of a GH/IGF-1 axis component-mediated signaling pathway.

The two-hybrid system is based on the modular nature of most transcription factors, which consist of separable DNA-binding and activation domains. Briefly, the assay utilizes two different DNA constructs. In one construct, the gene that codes for a GH/IGF-1 axis component protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: GH/IGF-1 axis component protein can be the fused to the activator domain.) If the “bait” and the “prey” proteins are able to interact, in vivo, forming a GH/IGF-1 axis component-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the GH/IGF-1 axis component protein. In another embodiment, the two-hybrid assay is used to monitor an interaction between two components of the axis that are known to interact. The two hybrid assay is conducted in the presence of a test compound, and the assay is used to determine whether the test compound enhances or diminishes the interaction between the components.

In another embodiment, modulators of a GH/IGF-I axis component gene expression are identified. For example, a cell or cell free mixture is contacted with a candidate compound and the expression of the GH/IGF-I axis component mRNA or protein evaluated relative to the level of expression of GH/IGF-I axis component mRNA or protein in the absence of the candidate compound. When expression of the GH/IGF-I axis component mRNA or protein is greater in the presence of the candidate compound than in its absence, the candidate compound is identified as a stimulator of GH/IGF-I axis component mRNA or protein expression. Alternatively, when expression of the GH/IGF-I axis component mRNA or protein is less (statistically significantly less) in the presence of the candidate compound than in its absence, the candidate compound is identified as an inhibitor of the GH/IGF-I axis component mRNA or protein expression. The level of the GH/IGF-I axis component mRNA or protein expression can be determined by methods for detecting GH/IGF-I axis component mRNA or protein.

Organismal Assays. Still other methods for evaluating a test compound include organismal based assays, e.g., using a mammal (e.g., a mouse, rat, primate, or other non-human), or other animal (e.g., Xenopus, zebrafish, or an invertebrate such as a fly or nematode). In some cases, the organism is a transgenic organism, e.g., an organism which includes a heterologous GH/IGF-1 axis component, (e.g., from a mammal, e.g., a human). The test compound can be administered to the organism once or as a regimen (regular or irregular). A parameter of the organism is then evaluated, e.g., an age-associated parameter or a parameter of the GH/IGF-1 axis. Test compounds that are indicated as of interest result in a change in the parameter relative to a reference, e.g., a parameter of a control organism. Other parameters (e.g., related to toxicity, clearance, and pharmacokinetics) can also be evaluated.

In some embodiment, the test compound is evaluated using an animal that has a particular disorder, e.g., a disorder described herein, e.g., an age-related disorder, a geriatric disorder, a neoplastic disorder, a non-neoplastic disorder, a metabolic disorder, an immunological disorder, a neurological disorder, a dermatological disorder, a dermatological tissue condition, or a cardio-vascular disorder. These disorders can also provide a sensitized system in which the test compound's effects on physiology can be observed. Exemplary disorders include: denervation, disuse atrophy; metabolic disorders (e.g., disorder of obese and/or diabetic animals such as db/db mouse and ob/ob mouse); cerebral, liver ischemia; cisplatin/taxol/vincristine models; various tissue (xenograph) transplants; transgenic bone models; Pain syndromes(include inflammatory and neuropathic disorders); Paraquat, genotoxic, oxidative stress models; pulmonary obstruction (e.g., asthma models); and tumor models. In a preferred embodiment, the animal model is an animal that has an altered phenotype when calorically restricted. For example, F344 rats provide a useful assay system for evaluating a test compound. When calorically restricted, F344 reats have a 0 to 10% incidence of nephropathy. However, when fed ad libitum, they have a 60 to 100% incidence of nephropathy. See Table 2.

TABLE 2
F344 rats - Frequency of nephropathy.

| Months | Ad lib | CR |
|---|---|---|
| 6 | 0% | 0% |
| 12 | 60% | 0% |
| 18 | 100% | 0% |
| 24 | 100% | 0% |

Additional animals are listed in Table 3: Exemplary Animal Models:

TABLE 3
Exemplary Animal Models

Mean
Lifespan
(months)

| Model | Ad lib | CR | Predisposition |
|---|---|---|---|
| SH Rat | 18 | 30 | Hypertension |
| SA Mouse | 10 | 15 | Amyloid |
| NZB Mouse | 12 | 16 | SLE |
| kd/kd Mouse | 8 | 18 | Nephritis |
| MRL/1 Mouse | 6 | >15 | Autoimmune |
| ob/ob Mouse | 14 | 26 | Diabetes |

To evaluate a test compound, it is administered to the animal (e.g., an F344 rat or an animal listed in Table 3), and a parameter of the animal is evaluated, e.g., after a period of time. The animal can be fed ad libitum or normally (e.g., not under caloric restriction, although some parameters can be evaluated under such conditions). Typically, a cohort of such animals is used for the assay. Generally, a test compound can be indicated as favorably altering lifespan regulation in the animal if the test compound affects the parameter in the direction of the phenotype of a similar animal subject to caloric restriction. Such test compounds may cause at least some of the lifespan regulatory effects of caloric restriction, e.g., a subset of such effects, without having to deprive the organism of caloric intake.

In one embodiment, the parameter is an age-associated or disease associated parameter, e.g., a symptom of the disorder associated with the animal model (e.g., the disorder in the “Predisposition column of Table 3). For example, the test compound can be administered to the SH Rat, and blood pressure is monitored. A test compound that is favorably indicated can cause an amelioration of the symptom relative to a similar reference animal not treated with the compound. In a related embodiment, the parameter is a parameter of the GH/IGF-1 axis. In some embodiment, a parameter relevant to a disorder or to aging can include: antioxidant levels (e.g., antioxidant enzyme levels or activity), stress resistance (e.g., paraquat resistance), core body temperature, glucose levels, insulin levels, thyroid-stimulating hormone levels, prolactin levels, and leutinizing hormone levels.

Still other in vivo models and organismal assays include evaluating an animal for a metabolic parameter, e.g., a parameter relevant to an insulin disorder. Exemplary metabolic parameters include: glucose concentration, insulin concentration, and insulin sensitivity. Another set of metabolic parameters are parameters associated with the function of the growth hormone (GH)/insulin-like growth factor (IGF-1) axis, e.g., GH concentration, IGF-1 concentration, GHS concentration, and so forth. Another exemplary system features tumors, e.g., in an animal model. The tumors can be spontaneous or induced. For example, the tumors can be developed from cells that have a variety of genetic constitutions, e.g., they can be p53+ or p53−. It is also possible to use organisms that an autoimmune disorder, e.g., an NZB mouse, which is predisposed to SLE. To evaluate features of bone disease, it is possible, for example, to use an animal that has an ovariectomy as a model,. e.g., for osteoporosis. Similarly, for joint disease, the model can be based on adjuvant arthritis (e.g., mice can be immunized with cartilege proteoglycans, high mobility group proteins, streptococcal cell wall material, or collegens); for kidney disease, kd/kd mice can be used. Animal models of cognition, particularly learning and memory are also available. Animal models of diabetes and its complications are also available, e.g., the streptozotocin model. Canine models can be used, for example, for evaluating stroke and ischemia.

In assessing whether a test compound is capable of inhibiting the GH/IGF-1 axis for the purpose of altering life span regulation, a number of age-associated parameters or biomarkers can be monitored or evaluated. Exemplary age associated parameters include: (i) lifespan of the cell or the organism; (ii) presence or abundance of a gene transcript or gene product in the cell or organism that has a biological age-dependent expression pattern; (iii) resistance of the cell or organism to stress; (iv) one or more metabolic parameters of the cell or organism (exemplary parameters include circulating insulin levels, blood glucose levels; fat content; core body temperature and so forth); (v) proliferative capacity of the cell or a set of cells present in the organism; and (vi) physical appearance or behavior of the cell or organism.

The term “average lifespan” refers to the average of the age of death of a cohort of organisms. In some cases, the “average lifespan” is assessed using a cohort of genetically identical organisms under controlled environmental conditions. Deaths due to mishap are discarded. For example, with respect to a nematode population, hermaphrodites that die as a result of the “bag of worms” phenotype are typically discard. Where average lifespan cannot be determined (e.g., for humans) under controlled environmental conditions, reliable statistical information (e.g., from actuarial tables) for a sufficiently large population can be used as the average lifespan.

Characterization of molecular differences between two such organisms, e.g., one reference organism and one organism treated with a GH/IGF-1 axis modulator can reveal a difference in the physiological state of the organisms. The reference organism and the treated organism are typically the same chronological age. The term “chronological age” as used herein refers to time elapsed since a preselected event, such as conception, a defined embryological or fetal stage, or, more preferably, birth. A variety of criteria can be used to determine whether organisms are of the “same” chronological age for the comparative analysis. Typically, the degree of accuracy required is a function of the average lifespan of a wildtype organism. For example, for the nematode C. elegans, for which the laboratory wildtype strain N2 lives an average of about 16 days under some controlled conditions, organisms of the same age may have lived for the same number of days. For mice, organism of the same age may have lived for the same number of weeks or months; for primates or humans, the same number of years (or within 2, 3, or 5 years); and so forth. Generally, organisms of the same chronological age may have lived for an amount of time within 15, 10, 5, 3, 2 or 1% of the average lifespan of a wildtype organism of that species. In a preferred embodiment, the organisms are adult organisms, e.g. the organisms have lived for at least an amount of time in which the average wildtype organism has matured to an age at which it is competent to reproduce.

In some embodiments, the organismal screening assay is performed before the organisms exhibit overt physical features of aging. For example, the organisms may be adults that have lived only 10, 30, 40, 50, 60, or 70% of the average lifespan of a wildtype organism of the same species.

Age-associated changes in metabolism, immune competence, and chromosomal structure have been reported. Any of these changes can be evaluated, either in a test subject (e.g., for an organism based assay), or for a patient (e.g., prior, during or after treatment with a therapeutic described herein.

In another embodiment, a marker associated with caloric restriction is evaluated in a subject organism of a screening assay (or a treated subject). Although these markers may not be age-associated, they may be indicative of a physiological state that is altered when the GH/IGF-1 axis is modulated. The marker can be an mRNA or protein whose abundance changes in calorically restricted animals. WO 01/12851 and U.S. Pat. No. 6,406,853 describe exemplary markers.

Cellular models derived from cells of an animal described herein or analogous to an animal model described herein can be used for a cell-based assay.

Animal models that can be used to evaluate aspects of Alzheimer's disease and neurodegenerative disorders that are caused at least in part by polyglutamine aggregation are provided further below.

Cells and animals for evaluating the effect of a compound on ALS status include a mouse which has an altered SOD gene, e.g., a SOD1-G93A transgenic mouse which carries a variable number of copies of the human G93A SOD mutation driven by the endogenous promoter, a SOD1-G37R transgenic mouse (Wong et al., Neuron, 14(6):1105-16 (1995)); SOD1-G85R transgenic mouse (Bruijn et al., Neuron, 18(2):327-38 (1997)); C. elegans strains expressing mutant human SOD1 (Oeda et al., Hum Mol Genet., 10:2013-23 (2001)); and a Drosophila expressing mutations in Cu/Zn superoxide dismutase (SOD). (Phillips et al., Proc. Nat]. Acad. Sci. U.S.A., 92:8574-78 (1995) and McCabe, Proc. Natl. Acad. Sci. U.S.A., 92:8533-34 (1995)).

Models for evaluating the effect of a test compound on muscle atrophy include, e.g., use of include: 1) rat medial gastrocnemius muscle mass loss resulting from denervation, e.g., by severing the right sciatic nerve at mid-thigh; 2) rat medial gastrocnemius muscle mass loss resulting from immobilization, e.g., by fixed the right ankle joint at 90 degrees of flexion; 3) rat medial gastrocnemius muscle mass loss resulting from hindlimb suspension; (see, e.g., U.S. Pat. No. 2003-0,129,686); 4) skeletal muscle atrophy resulting from treatment with the cachectic cytokine, interleukin-1 (IL-1) (R. N. Cooney, S. R. Kimball, T. C. Vary, Shock 7, 1-16 (1997)); and 5) skeletal muscle atrophy resulting from treatment with the glucocorticoid, dexamethasone (A. L. Goldberg, J Biol Chem 244, 3223-9 (1969).).Models 1, 2, and 3 induce muscle atrophy by altering the neural activity and/or external load a muscle experiences to various degrees. Models 4 and 5 induce atrophy without directly affecting those parameters.MS (experimental autoimmune encephalomyelitis (EAE)), e.g., as described by Goverman et al., Cell. 72:551-60 (1993), and primate models as reviewed by Brok et al., Immunol. Rev., 183:173-85 (2001).

Exemplary animal models for AMD (age-related macular degeneration) include: laser-induced mouse model simulating exudative (wet) macular degeneration Bora et al., Proc. Natl. Acad. Sci. U S A., 100:2679-84 (2003); a transgenic mouse expressing a mutated form of cathepsin D resulting in features associated with the “geographic atrophy” form of AMD (Rakoczy et al., Am. J. Pathol., 161:1515-24 (2002)); and a transgenic mouse overexpressing VEGF in the retinal pigment epithelium resulting in CNV. Schwesinger et al., Am. J. Pathol. 158:1161-72 (2001).

Exemplary animal models of Parkinson's disease include primates rendered parkinsonian by treatment with the dopaminergic neurotoxin 1-methyl-4 phenyl 1,2,3,6-tetrahydropyridine (MPTP) (see, e.g., U.S. appl Ser. No. 20,030,055,523 and Wichmann et al., Ann. N.Y. Acad. Sci., 991:199-213 (2003); 6-hydroxydopamine-lesioned rats (e.g., Lab. Anim. Sci.,49:363-71 (1999)); and transgenic invertebrate models (e.g., Lakso et al., J. Neurochem., 86:165-72 (2003) and Link, Mech. Ageing Dev., 122:1639-49 (2001)).

Exemplary molecular models of Type II diabetes include: a transgenic mouse having defective Nkx-2.2 or Nkx-6.1; (U.S. Pat. No. 6,127,598); Zucker Diabetic Fatty fa/fa (ZDF) rat. (U.S. Pat. No. 6,569,832); and Rhesus monkeys, which spontaneously develop obesity and subsequently frequently progress to overt type 2 diabetes (Hotta et al., Diabetes, 50:1126-33 (2001); and a transgenic mouse with a dominant-negative IGF-I receptor (KR-IGF-IR) having Type 2 diabetes-like insulin resistance.

Exemplary animal and cellular models for neuropathy include: vincristine induced sensory-motor neuropathy in mice (U.S. appl Ser. No. 5,420,112) or rabbits (Ogawa et al., Neurotoxicology, 21:501-11 (2000)); a streptozotocin (STZ)-diabetic rat for study of autonomic neuropathy (Schmidt et al., Am. J. Pathol., 163:21-8 (2003)); and a progressive motor neuropathy (pmn) mouse (Martin et al., Genomics, 75:9-16 (2001)).

Antibodies

Immunoglobulins can also be produced that bind to a GH/IGF-1 axis component and, for example, reduce axis activity. For example, an immunoglobulin can bind to a receptor and modulate receptor activity or ability of ligand to interact or modulate the receptor. For example, an immunoglobulin can bind to GH receptor and prevent GH binding, without itself activating the receptor. Other receptors such as IGF1-R, GHS-R, and GHRH-R as well as the insulin receptor and IRS can also be antagonized using an immunoglobulin. Similarly, an immunoglobulin can bind to a secreted axis component, e.g., GH, IGF-1, GHS (e.g., ghrelin)and so forth. In a preferred embodiment, the immunoglobulin is human, humanized, deimmunized, or otherwise non-antigenic in the subject.

An immunoglobulin can be, for example, an antibody or an antigen-binding fragment thereof. As used herein, the term “immunoglobulin” refers to a protein consisting of one or more polypeptides that include one or more immunoglobulin variable domain sequences. A typical immunoglobulin includes at least a heavy chain immunoglobulin variable domain and a light chain immunoglobulin variable domain. An immunoglobulin protein can be encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids). The term “antigen-binding fragment” of an antibody (or simply “antibody portion,” or “fragment”), as used herein, refers to one or more fragments of a full-length antibody that retain the ability to specifically bind to the antigen. Examples of antigen-binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

In one embodiment, the antibody against the axis component is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey). Preferably, the non-human antibody is a rodent (mouse or rat antibody). Method of producing rodent antibodies are known in the art. Human monoclonal antibodies can be generated using transgenic mice carrying the human immunoglobulin genes rather than the mouse system (see, e.g., WO 91/00906 and WO 92/03918). Other methods for generating immunoglobulin ligands include phage display (e.g., as described in U.S. Pat. No. 5,223,409 and WO 92/20791).

RNAi

It is also possible to attenuate GH/IGF-1 axis activity using a double-stranded RNA (dsRNA) that mediates RNA interference (RNAi). The dsRNA can be delivered to cells or to an organism. Endogenous components of the cell or organism can trigger RNA interference (RNAi) which silences expression of genes that include the target sequence. dsRNA can be produced by transcribing a cassette in both directions, for example, by including a T7 promoter on either side of the cassette. The insert in the cassette is selected so that it includes a sequence from a GH/IGF-1 axis component to be attenuated. The sequence need not be full length, for example, an exon, or at least 50 nucleotides, preferably from the 5′ half of the transcript, e.g., within 300 nucleotides of the ATG. See also, the HiScribe™ RNAi Transcription Kit (New England Biolabs, Mass.) and Fire, A. (1999) Trends Genet. 15, 358-363. dsRNA can be digested into smaller fragments. See, e.g., U.S. patent application Ser. No. 2002-0,086,356.

dsRNAs can be used to silence gene expression in mammalian cells. See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature. 411(6836):494-8; Yang, D. et al. (2002) Proc. Natl. Acad. Sci. USA 99, 9942-9947.

For example, double stranded RNA molecules complementary to a nucleic acid encoding GHRH, GHRH-R, GHS-R, GH, GH-R, IGF-1, IGF-1-R, PI(3) kinase, PDK1, or an AKT protein (e.g., AKT-1, -2, or -3) can be used to attenuate activity of the GH/IGF-1 axis.

In one embodiment, an siRNA is used. siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature. 411(6836):494-8; Yang, D. et al. (2002) Proc. Natl. Acad. Sci. USA 99, 9942-9947, U.S. Pat. No. 20,030,166,282 and 20,030,143,204.

Stem Cell Therapy

It is also possible to modify stem cells using nucleic acid recombination, e.g., to insert a transgene. The modified stem cell can be administered to a subject. Methods for cultivating stem cells in vitro are described, e.g., in U.S. application Ser. No. 2002-0,081,724. In some examples, the stem cells can be induced to differentiate in the subject and express the transgene.

Pharmaceutical Compositions

A compound that modulates the GH/IGF-1 axis can be incorporated into a pharmaceutical composition for administration to a subject, e.g., a human, a non-human animal, e.g., an animal patient (e.g., pet or agricultural animal) or an animal model (e.g., an animal model for aging or a metabolic disorder (e.g., a disorder of the GH/IGF-1 axis or a pancreatic or insulin related disorder). Such compositions typically include the a small molecule (e.g., a small molecule that is a GH/IGF-1 antagonist), nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Exemplary compounds that can be used for treatment include: Pegvisomant, a somatostatin agonist (such as L-054,522), an IGF-1R competitive inhibitor such as Tyrphostin A G 538 (see, e.g., Biochemistry 2000.39.15705) or Tyrphostin AG 1024 (Br J Cancer Dec. 14, 2001;85(12):2017-21) an IGF-1R antagonist such as H-1356 (see, e.g., Diabetes Res Clin Pract February 2002;55(2):89-98) and hetero-aryl-aryl ureas (see, e.g., U.S. Pat. No. 6,337,338), a Akt modulator such as trisenox (see, e.g., Blood 2001, 98:618) or UCN-01 (e.g., mediating desphosphorylation and inactivation of Akt; Oncogene 2002.21.1727), or a PI(3) kinase inhibitor, e.g., LY294002 (Mol Endocrinol February 2002;16(2):342-52) or Wortmannin (see, e.g., J Cell Biochem 2002;84:708-16), a GHRH antagonist peptide such as JV-1-36, JV-1-38 (Proc. Natl. Acad. Sci. USA 1999 96:692); a GHRH/GHRH receptor antagonist such as GHRH-44 (see, e.g., J Clin Endocrinol Metab November 2001; ;86(11):5485-90); an inhibitor of GH release such as CST-14 (cortistatin-14); Sandostatin LAR; a somatostatin-analogist cyclic peptide e.g., as described in U.S. Pat. No. 5,962,409; octreotide acetate; slow release analog of somatostatin such as SR-lancreotide, BIM 23014 or another compound, e.g., a compound described herein.

TABLE 4
Exemplary Compounds

| Description | Compound | Source |
|---|---|---|
| Somatostatin-analogous | | Zentaris |
| cyclic peptides | | |
| with inhibitory activity on GH | | |
| IGF-1 receptor antagonist | H-1356 cyclic | Bachem Bioscience |
| | peptide, C-T-A- | |
| | A-P-L-K-P-A-K-S-C- | |
| Inhibitor of IGF-1R | Tyrphostin AG 1024 | Alexis Biochemicals, Calbiochem |
| GHRH receptor antagonist | GHRH antagonist | GHRH antagonist from |
| | and GHRH-44 | Bachem Bioscience; |
| | | GHRH-44 from |
| | | Peninsula Laboratories |
| GH receptor antagonist | pegvisomant | Pharmacia |
| IGF-1R antagonists | heteroaryl-aryl ureas | Telik, Inc. |
| Janus-kinase-3 inhibitor | WHI-P154 | Calbiochem #420104 |
| dephosphorylation and | UCN-01 - 7- | Kyowa Hakko |
| inactivation of Akt | hydroxystaurosporine | |
| IGF-1R competitive inhibitor | tyrphostin AG 538 | Calbiochem AG538 |
| | | Cat #658403, I-OMe 538 Cat#658417 |
| Inhibitor of GH release in rats | CST-14 (cortistatin-14) | Penlabs, CAT. No. 8027 |
| Sandostatin LAR | octreotide acetate | Novartis; Penlabs - CAT. No. 8060 |
| AKT inhibitor | trisenox | Marketer - Cell Therapeutics |
| Modulator of GH release | Somatostatin | Somatostatins from |
| | | Peninsula Labs (Penlabs) |
| slow release analog of | SR-lancreotide, BIM 23014 | Beaufour Ipsen |
| somatostatin GHRH | JV-1-36, JV-1-38 | Phoenix peptide |
| antagonist peptides | | |

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells, e.g., a pituitary cell) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some implementations, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments.

For antibody compounds that modulate the axis, one preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule may depend upon a number of factors, such as the potency of the small molecule with respect to the expression or activity to be modulated (e.g., affinity for target compound and efficacy) and pharmacokinetic properties. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules that modulate the GH/IGF-1 axis can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

A modulator of the GH/IGF-1 axis that alters lifespan regulation or at least one symptom of aging or an age related disease, e.g., a modulator described herein, can be provided in a kit. The kit includes (a) the modulator, e.g., a composition that includes the modulator, and (b) informational material. The informational material can be descriptive, instructional, marketing or other material that relates to the methods described herein and/or the use of the modulator for the methods described herein. For example, the informational material describes methods for administering the modulator to alter lifespan regulation or at least one symptom of aging or an age related disease.

In one embodiment, the informational material can include instructions to administer the modulator in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. For example, the human is an adult, e.g., an adult with normal GH/IGF-1 axis activity for the adult's age, or with abnormal axis activity (e.g., above average activity for the adult's age). The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the modulator, the composition of the kit can include other ingredients, such as a solvent or buffer, a stabilizer or a preservative, and/or a second agent for treating a condition or disorder described herein, e.g. an age-related disorder. Alternatively, the other ingredients can be included in the kit, but in different compositions or containers than the modulator. In such embodiments, the kit can include instructions for admixing the modulator and the other ingredients, or for using the modulator together with the other ingredients.

The modulator can be provided in any form, e.g., liquid, dried or lyophilized form. It is preferred that the modulator be substantially pure and/or sterile. When the modulator is provided in a liquid solution, the liquid solution preferably is an aqueous solution, with a sterile aqueous solution being preferred. When the modulator is provided as a dried form, reconstitution generally is by the addition of a suitable solvent. The solvent, e.g., sterile water or buffer, can optionally be provided in the kit.

The kit can include one or more containers for the composition containing the modulator. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the modulator. For example, the kit includes a plurality of syringes, ampules, foil packets, or blister packs, each containing a single unit dose of the modulator. The containers of the kits can be air tight and/or waterproof.

The compositions can be administered to a subject, e.g., an adult subject, particularly a healthy adult subject or a subject having an age-related disease. In the latter case, the method can include evaluating a subject, e.g., to characterize a symptom of an age-related disease or other disease marker, and thereby identifying a subject as having a neurodegenerative disease, a disease associated with protein misfolding or protein aggregation (e.g., other than polyglutamine aggregation) or an age-related disease or being pre-disposed to such a disease.

Alzheimer's Disease

Alzheimer's Disease (AD) is a complex neurodegenerative disease that results in the irreversible loss of neurons. It provides merely one example of a neurodegenerative disease that has symptoms caused at least in part by protein aggregation. Clinical hallmarks of Alzheimer's Disease include progressive impairment in memory, judgment, orientation to physical surroundings, and language. Neuropathological hallmarks of AD include region-specific neuronal loss, amyloid plaques, and neurofibrillary tangles. Amyloid plaques are extracellular plaques containing the β amyloid peptide (also known as Aβ, or Aβ42), which is a cleavage product of the β-amyloid precursor protein (also known as APP). Neurofibrillary tangles are insoluble intracellular aggregates composed of filaments of the abnormally hyperphosphorylated microtubule-associated protein, tau. Amyloid plaques and neurofibrillary tangles may contribute to secondary events that lead to neuronal loss by apoptosis (Clark and Karlawish, Ann. Intern. Med. 138(5):400-410 (2003). For example, β-amyloid induces caspase-2-dependent apoptosis in cultured neurons (Troy et al. J. Neurosci. 20(4):1386-1392). The deposition of plaques in vivo may trigger apoptosis of proximal neurons in a similar manner.

Mutations in genes encoding APP, presenilin-1, and presenilin-2 have been implicated in early-onset AD (Lendon et al. JAMA 227:825 (1997)). Mutations in these proteins have been shown to enhance proteolytic processing of APP via an intracellular pathway that produces Aβ. Aberrant regulation of Aβ processing may be central to the formation of amyloid plaques and the consequent neuronal damage associated with plaques.

A variety of criteria, including genetic, biochemical, physiological, and cognitive criteria, can be used to evaluate AD in a subject. Symptoms and diagnosis of AD are known to medical practitioners. Some exemplary symptoms and markers of AD are presented below. Information about these indications and other indications known to be associated with AD can be used as an “AD-related parameter.” An AD-related parameter can include qualitative or quantitative information. An example of quantitative information is a numerical value of one or more dimensions, e.g., a concentration of a protein or a tomographic map. Qualitative information can include an assessment, e.g., a physician's comments or a binary (“yes”/“no”) and so forth. An AD-related parameter includes information that indicates that the subject is not diagnosed with AD or does not have a particular indication of AD, e.g., a cognitive test result that is not typical of AD or a genetic APOE polymorphism not associated with AD.

Progressive cognitive impairment is a hallmark of AD. This impairment can present as decline in memory, judgment, decision making, orientation to physical surroundings, and language (Nussbaum and Ellis, New Eng. J. Med. 348(14): 1356-1364 (2003)). Exclusion of other forms of dementia can assist in making a diagnosis of AD.

Neuronal death leads to progressive cerebral atrophy in AD patients. Imaging techniques (e.g., magnetic resonance imaging, or computed tomography) can be used to detect AD-associated lesions in the brain and/or brain atrophy.

AD patients may exhibit biochemical abnormalities that result from the pathology of the disease. For example, levels of tau protein in the cerebrospinal fluid is elevated in AD patients (Andreasen, N. et al. Arch Neurol. 58:349-350 (2001)). Levels of amyloid beta 42 (Aβ42) peptide can be reduced in CSF of AD patients (Galasko, D., et al. Arch. Neurol. 55:937-945 (1998)). Levels of Aβ42 can be increased in the plasma of AD patients (Ertekein-Taner, N., et al. Science 290:2303-2304 (2000)). Techniques to detect biochemical abnormalities in a sample from a subject include cellular, immunological, and other biological methods known in the art. For general guidance, see, e.g., techniques described in Sambrook & Russell, Molecular Cloning: A Laboratory Manual, 3^{rd} Edition, Cold Spring Harbor Laboratory, N.Y. (2001), Ausubel et al., Current Protocols in Molecular Biology (Greene Publishing Associates and Wiley Interscience, N.Y. (1989), (Harlow, E. and Lane, D. (1988) Antibodies: A Laboratory Manual, Cold Spring Harbor Laboratory Press, Cold Spring Harbor, N.Y.), and updated editions thereof.

For example, antibodies, other immunoglobulins, and other specific binding ligands can be used to detect a biomolecule, e.g., a protein or other antigen associated with AD. For example, one or more specific antibodies can be used to probe a sample. Various formats are possible, e.g., ELISAs, fluorescence-based assays, Western blots, and protein arrays. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1.

Proteins can also be analyzed using mass spectroscopy, chromatography, electrophoresis, enzyme interaction or using probes that detect post-translational modification (e.g., a phosphorylation, ubiquitination, glycosylation, methylation, or acetylation).

Nucleic acid expression can be detected in cells from a subject, e.g., removed by surgery, extraction, post-mortem or other sampling (e.g., blood, CSF). Expression of one or more genes can be evaluated, e.g., by hybridization based techniques, e.g., Northern analysis, RT-PCR, SAGE, and nucleic acid arrays. Nucleic acid arrays are useful for profiling multiple mRNA species in a sample. A nucleic acid array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

Metabolites that are associated with AD can be detected by a variety of means, including enzyme-coupled assays, using labeled precursors, and nuclear magnetic resonance (NMR). For example, NMR can be used to determine the relative concentrations of phosphate-based compounds in a sample, e.g., creatine levels. Other metabolic parameters such as redox state, ion concentration (e.g., Ca²⁺)(e.g., using ion-sensitive dyes), and membrane potential can also be detected (e.g., using patch-clamp technology).

Information about an AD-associated marker can be recorded and/or stored in a computer-readable format. Typically the information is linked to a reference about the subject and also is associated (directly or indirectly) with information about the identity of one or more nucleotides in a gene that encodes an GH/IGF-1 axis component in the subject.

In one embodiment, a non-human animal model of AD (e.g., a mouse model) is used, e.g., to evaluate a compound or a therapeutic regimen. For example, U.S. Pat. No. 6,509,515 describes one such model animal which is naturally able to be used with learning and memory tests. The animal expresses an amyloid precursor protein (APP) sequence at a level in brain tissues such that the animal develops a progressive neurologic disorder within a short period of time from birth, generally within a year from birth, preferably within 2 to 6 months, from birth. The APP protein sequence is introduced into the animal, or an ancestor of the animal, at an embryonic stage, preferably the one cell, or fertilized oocyte, stage, and generally not later than about the 8-cell stage. The zygote or embryo is then developed to term in a pseudo-pregnant foster female. The amyloid precursor protein genes are introduced into an animal embryo so as to be chromosomally incorporated in a state which results in super-endogenous expression of the amyloid precursor protein and the development of a progressive neurologic disease in the cortico-limbic areas of the brain, areas of the brain which are prominently affected in progressive neurologic disease states such as AD. The gliosis and clinical manifestations in affected transgenic animals model neurologic disease. The progressive aspects of the neurologic disease are characterized by diminished exploratory and/or locomotor behavior and diminished 2-deoxyglucose uptake/utilization and hypertrophic gliosis in the cortico-limbic regions of the brain. Further, the changes that are seen are similar to those that are seen in some aging animals. Other animal models are also described in U.S. Pat. Nos. 5,387,742; 5,877,399; 6,358,752; and 6,187,992.

In one embodiment, the animal also includes a deficiency in at least one cell in a GH/IGF-1 axis component, e.g., a genetic mutation, an antisense construct, a construct that produces RNAi. The deficiency can also be produced epigenetically, e.g., by administering RNAi, e.g., siRNA.

In another embodiment, the animal is hypersensitized, e.g., because it has an enhanced level of GH/IGF-1 axis activity. This animal model can be used to screen for compounds which restore the level of activity to normal, e.g., produce the AD progression seen if the animal had an otherwise normal GH/IGF-1 axis. The animal can have enhanced levels of activity by pharmaceutical intervention, by genetic alteration (e.g., overproducing a positively acting axis component) or epigenetically (e.g., inhibiting an inhibitor of the axis) and so forth.

Amyotrophic Lateral Sclerosis (ALS; Lou Gehrig's Disease)

ALS refers to a class of disorders that comprise upper and lower motor neurons. The incidence of ALS increases substantially in older adults. These disorders are characterized by major pathological abnormalities include selective and progressive degeneration of the lower motor neurons in the spinal cord and the upper motor neurons in the cerebral cortex resulting in motor neuron death, which causes the muscles under their control to weaken and waste away leading to paralysis. Examples of ALS disorders include classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons) or familial ALS (a genetic version of ALS), or a combination of these conditions. (see, e.g., U.S. appl Ser. No. 20,020,198,236 and U.S. appl Ser. No. 20,030,130,357).

The ALS status of an individual may be evaluated by neurological examination or other means, such as MRI, FVC, MUNE etc. (see, e.g., U.S. appl Ser. No. 20,030,130,357). Symptoms include muscle weakness in the hands, arms, legs; swallowing or breathing difficulty; twitching (fasciculation) and cramping of muscles; and reduced use of the limbs. The invention includes administering an agent that modulates the IGF-1/GH axis in an amount effective to relieve one or more ALS symptoms, e.g., in an individual having, at risk to,

Methods for evaluating ALS status of an individual can include evaluating the “excitatory amino acid transporter type 2” (EAAT2) protein or gene, the Copper-Zinc Superoxide Dismutase (SOD1) protein or gene, mitochondrial Complex I activity, levels of polyamines, such as putraceine, spermine and spermidine, ornithine decarboxylase activity, and a gene that encodes a putative GTPase regulator (see Nat. Genet., 29(2): 166-73 (2001)).

Cells and animals for evaluating the effect of a compound on ALS status include a mouse which has an altered SOD gene, e.g., a SOD 1-G93A transgenic mouse which carries a variable number of copies of the human G93A SOD mutation driven by the endogenous promoter, a SOD1-G37R transgenic mouse (Wong et al., Neuron, 14(6):1105-16 (1995)); SOD1-G85R transgenic mouse (Bruijn et al., Neuron, 18(2):327-38 (1997)); C. elegans strains expressing mutant human SOD1 (Oeda et al., Hum Mol Genet., 10:2013-23 (2001)); and a Drosophila expressing mutations in Cu/Zn superoxide dismutase (SOD). (Phillips et al., Proc. Natl. Acad. Sci. U.S.A., 92:8574-78 (1995) and McCabe, Proc. Natl. Acad. Sci. U.S.A., 92:8533-34 (1995)).

Skeletal Muscle Atrophy

Muscle atrophy includes numerous neuromuscular, metabolic, immunological and neurological disorders and diseases as well as starvation, nutritional deficiency, metabolic stress, diabetes, aging, muscular dystrophy, or myopathy. Muscle atrophy occurs during the aging process. Muscle atrophy also results from reduced use or disuse of the muscle. Symptoms include a decline in skeletal muscle tissue mass. In human males, muscle mass declines by one-third between the ages of 50 and 80.

Some molecular features of muscle atrophy include the upregulation of ubiquitin ligases, and the loss of myofibrillar proteins (Furuno et al., J. Biol. Chem., 265:8550-8557, 1990). The breakdown of these proteins can be followed, e.g., by measuring 3-methyl-histidine production, which is a specific constituent of actin, and in certain muscles of myosin (Goodman, Biochem. J, 241:121-12, 1987 and Lowell, et al., Metabolism, 35:1121-112, 1986; Stein and Schluter, Am. J. Physiol. Endocrinol. Metab. 272: E688-E696, 1997). Release of creatine kinase (a cell damage marker) (Jackson, et al., Neurology, 41: 101 104, 1991) can also be indicative.

Multiple Sclerosis

Multiple sclerosis (MS) is a neuromuscular disease characterized by focal inflammatory and autoimmune degeneration of cerebral white matter. White matter becomes inflamed, and inflammation is followed by destruction of myelin (forming “lesions” which are marked by an infiltration of numerous immune cells, especially T-cell lymphocytes and macrophages. MS can cause a slowing or complete block of nerve impulse transmission and, thus, diminished or lost bodily function. A patient who has MS may have one of a variety of grade of MS (e.g., relapsing-remitting MS, primary progressive MS, secondary progressive, and Marburg's variant MS).

Symptoms can include vision problems such as blurred or double vision, red-green color distortion, or even blindness in one eye, muscle weakness in the extremities, coordination and balance problems, muscle spasticity, muscle fatigue, paresthesias, fleeting abnormal sensory feelings such as numbness, prickling, or “pins and needles” sensations, and in the worst cases, partial or complete paralysis. About half of the people suffering from MS also experience cognitive impairments, such as for example, poor concentration, attention, memory and/or judgment. (see, e.g., U.S. Pat. Nos. 2003-0,130,357 and 2003-0,092,089)

Molecular markers of MS include a number of genetic factors, e.g., Caucasian haplotype DRB*1501-DQA1*0102-DQB1*0602 (U.S. appl Ser. No. 20,030,113,752), a point mutation in the protein tyrosine phosphatase receptor-type C. (U.S. appl Ser. No. 20,030,113,752), absence of wild-type SARG-1-protein, presence of mutated SARG-1-protein, or absence or mutation in the nucleic acids encoding wild-type SARG-1. (see, e.g., U.S. appl Ser. No. 20,030,113,752) and protein indicators, e.g., Myelin Basic Protein auto-antibody in cerebrospinal fluid. (see, e.g., U.S. appl Ser. No. 20,030,092,089)

Cellular and animal models of MS include transgenic mouse model for chronic MS (experimental autoimmune encephalomyelitis (EAE)), e.g., as described by Goverman et al., Cell. 72:551-60 (1993), and primate models as reviewed by Brok et al., Immunol. Rev., 183:173-85 (2001).

Age-Related Macular Degeneration (AMD)

Macular degeneration includes a variety of diseases characterized by a progressive loss of central vision associated with abnormalities of Bruch's membrane and the retinal pigment epithelium. (see, e.g., U.S. appl Ser. No. 20,030,138,798). AND occurs in 1.2% of the population between 52 and 64 years of age and 20% of patients over the age of 75. (see, e.g., U.S. appl Ser. No. 20,030,087,889) Macular degeneration occurs in two forms, “atrophic” (“non-exudative” or “dry” form) and “exudative” (“wet” form). A less common form of AMD is “atrophic AMD,” which is due to dead RPE cells. (U.S. application Ser. No. 20,030,093,064).

Symptoms of AMD include: straight lines in the field of vision appear wavy; type in books, magazines and newspapers appears blurry; and dark or empty spaces block the center of vision. (see, e.g., U.S. appl Ser. No. 20,030,065,020)

Exemplary molecular markers that can be used to evaluate an AMD status include: the nucleic acid sequence of a gene encoding FBNL or the amino acid sequence of the FBNL protein: 345 Arg>Trp and 362 Arg>Gln; (see, e.g., U.S. appl Ser. No. 20,030,138,798); increases in the pigment A2E, N-retinyl-N-retinylidene ethanolamine, ultimately leading to release of cytochrome c into the cytoplasm (U.S. appl Ser. No. 20,030,050,283); auto-antibodies against various macular degeneration-associated molecules including fibulin-3, vitronectin, β-crystallin A2, β-crystallin A3, β-crystallin A4, β-crystallin S, calreticulin, 14-3-3 protein epsilon, serotransferrin, albumin, keratin, pyruvate carboxylase, or villin 2 (see, e.g., U.S. appl Ser. No. 20,030,017,501); abnormal activity or level of complement pathway molecules including clusterin, C6 or C5b-9 complex (see, e.g., U.S. appl Ser. No. 20,020,015,957); and accumulation of the pigment lipofuscin in lysosomes of retinal pigment epithelial (RPE) cells (Suter et al., J Biol Chem. 275:39625-30 (2000)).

Parkinson's Disease

Parkinson's disease includes Neurodegeneration of dopaminergic neurons in the substantia nigra resulting in the degeneration of the nigrostriatal dopamine system that regulates motor function. This pathology, in turn, leads to motor dysfunctions. (see, e.g., and Lotharius et al., Nat. Rev. Neurosci., 3:932-42 (2002).) Exemplary motor symptoms include: akinesia, stooped posture, gait difficulty, postural instability, catalepsy, muscle rigidity, and tremor. Exemplary non-motor symptoms include: depression, lack of motivation, passivity, dementia and gastrointestinal dysfunction (see, e.g., Fahn, Ann. N.Y. Acad. Sci., 991:1-14 (2003) and Pfeiffer, Lancet Neurol., 2:107-16 (2003)) Parkinson's has been observed in 0.5 to 1 percent of persons 65 to 69 years of age and 1 to 3 percent among persons 80 years of age and older. (see, e.g., Nussbaum et al., N. Engl. J. Med., 348:1356-64 (2003))

Molecular markers of Parkinson's disease include reduction in aromatic L-amino acid decarboxylase (AADC). (see, e.g., U.S. appl Ser. No. 20,020,172,664); loss of dopamine content in the nigrostriatal neurons (see, e.g., Fahn, Ann. N.Y. Acad. Sci., 991:1-14 (2003) and Lotharius et al., Nat. Rev. Neurosci., 3:932-42 (2002)). In some familial cases, PD is linked to mutations in single genes encoding alpha-synuclein and parkin (an E3 ubiquitin ligase) proteins. (e.g., Riess et al., J. Neurol. 250 Suppl 1:I3-10 (2003) and Nussbaum et al., N. Engl. J. Med., 348:1356-64 (2003)). A missense mutation in a neuron-specific C-terminal ubiquitin hydrolase gene is also associated with Parkinson's. (e.g., Nussbaum et al., N. Engl. J. Med., 348:1356-64 (2003))

Non-Insulin-Dependent Diabetes

Non-insulin-dependent Diabetes is also called “adult onset” diabetes and Type 2 diabetes. Type 2 diabetes also includes “non-obese type 2” and “obese type 2.” Type II diabetes can be characterized by (1) reduced pancreatic-beta-islet-cell secretion of insulin such that less than necessary amounts of insulin are produced to keep blood glucose levels in balance and/or (2) “insulin resistance,” wherein the body fails to respond normally to insulin. (U.S. Pat. No. 5,266,561 and U.S. Pat. No. 6,518,069). For example, glucose-stimulated insulin levels typically fail to rise above 4.0 nmol/L. (U.S. Pat. No. 5,266,561) Exemplary symptoms of Type II diabetes include: hyperglycemia while fasting (U.S. Pat. No. 5,266,561); fatigue; excessive thirst; frequent urination; blurred vision; and an increased rate of infections.

Molecular indications of Type II diabetes include islet amyloid deposition in the pancreases.

Neuropathy

A neuropathy can include a central and/or peripheral nerve dysfunction caused by systemic disease, hereditary condition or toxic agent affecting motor, sensory, sensorimotor or autonomic nerves. (see, e.g., U.S. appl Ser. No. 20,030,013,771)

Symptoms can vary depending upon the cause of the nerve damage and the particular types of nerves affected. For example, symptoms of motor neuropathy include clumsiness in performing physical tasks or as muscular weakness, exhaustion after minor exertion, difficulty in standing or walking and attenuation or absence of a neuromuscular reflex. (U.S. app Ser. No. 20,030,013,771) symptoms of autonomic neuropathy include constipation, cardiac irregularities and attenuation of the postural hypotensive reflex. (U.S. app Ser. No. 20,030,013,771), symptoms of sensory neuropathy include pain and numbness; tingling in the hands, legs or feet; and extreme sensitivity to touch, and symptoms of retinopathy include blurred vision, sudden loss of vision, black spots, and flashing lights.

Guillain-Barr syndrome is a type of motor neuropathy that usually occurs two to three weeks after a flu-like disease or other infection. Symptoms include ascending weakness wherein weakness begins in the lower extremities and ascends to the upper extremities. An elevation of the protein level in the spinal fluid without an increase in the number of white cells also results. (U.S. appl Ser. No. 20,030,083,242)

Evaluating Polyglutamine Aggregation

A variety of cell free assays, cell based assays, and organismal assays are available for evaluating polyglutamine aggregation, e.g., Huntingtin polyglutamine aggregation. Some examples are described, e.g., in U.S. Pat. No. 2003-0,109,476.

Assays (e.g., cell free, cell-based, or organismal) can include a reporter protein that includes a polyglutamine repeat region which has at least 35 polyglutamines. The reporter protein can be easily detectable, e.g., by fluorescence. For example, the protein is conjugated to a fluorophore, for example, fluorescein isothiocyanate (FITC), allophycocyanin (APC), R-phycoerythrin (PE), peridinin chlorophyll protein (PerCP), Texas Red, Cy3, Cy5, Cy7, or a fluorescence resonance energy tandem fluorophore such as PerCP-Cy5.5, PE-Cy5, PE-Cy5.5, PE-Cy7, PE-Texas Red, and APC-Cy7. In another example the protein is “intrinsically fluorescent” in that it has a chromophore is entirely encoded by its amino acid sequence and can fluoresce without requirement for cofactor or substrate. For example, the protein can include a green fluorescent protein (GFP)-like chromophore. As used herein, “GFP-like chromophore” means an intrinsically fluorescent protein moiety comprising an 11-stranded β-barrel with a central α-helix, the central α-helix having a conjugated π-resonance system that includes two aromatic ring systems and the bridge between them.

The GFP-like chromophore can be selected from GFP-like chromophores found in naturally occurring proteins, such as A. victoria GFP (GenBank accession number AAA27721), Renilla renifonnis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation. The methods for engineering such modified GFP-like chromophores and testing them for fluorescence activity, both alone and as part of protein fusions, are well-known in the art. A variety of such modified chromophores are now commercially available and can readily be used in the fusion proteins of the present invention. For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins that further include a polyglutamine region. A variety of EGFP vectors, both plasmid and viral, are available commercially (Clontech Labs, Palo Alto, Calif., USA). Still other engineered GFP proteins are known. See, e.g., Heim et al., Curr. Biol. 6:178-182 (1996); Cormack et al., Gene 173:33-38 (1996), BFP2, EYFP (“enhanced yellow fluorescent protein”), EBFP, Ormo et al., Science 273:1392-1395 (1996), Heikal et al., Proc. Natl. Acad. Sci. USA 97:11996-12001(2000). ECFP (“enhanced cyan fluorescent protein”) (Clontech Labs, Palo Alto, Calif., USA). The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048.

In one embodiment, a reporter protein that includes a polyglutamine repeat region which has at least 35 polyglutamines is used in a cell-based assay.

In one example, PC12 neuronal cell lines that have a construct engineered to express a protein encoded by HD gene exon 1 containing alternating, repeating codons fused to an enhanced GFP (green fluorescent protein) gene can be used. See, e.g., Boado et al. J. Pharmacol and Experimental Therapeutics 295(1): 239-243 (2000) and Kazantsev et al. Proc. Natl. Acad. Sci. USA 96: 11404-09 (1999). Expression of this gene leads to the appearance of green fluorescence co-localized to the site of protein aggregates. The HD gene exon 1-GFP fusion gene is under the control of an inducible promoter regulated by muristerone. A particular construct has approximately 46 glutamine repeats (encoded by either CAA or CAG). Other constructs have, for example, 103 glutamine repeats. PC12 cells are grown in DMEM, 5% Horse serum (heat inactivated), 2.5% FBS and 1% Pen-Strep, and maintained in low amounts on Zeocin and G418. The cells are plated in 24-well plates coated with poly-L-lysine coverslips, at a density of 5·10⁵ cells/ml in media without any selection. Muristerone is added after the overnight incubation to induce the expression of HD gene exon 1-GFP. The cells can be contacted with a test compound, e.g., before or after plating and before or after induction. The data can be acquired on a Zeiss inverted 100M Axioskop equipped with a Zeiss 510 LSM confocal microscope and a Coherent Krypton Argon laser and a Helium Neon laser. Samples can be loaded into Lab-Tek II chambered coverglass system for improved imaging. The number of Huntingtin-GFP aggregations within the field of view of the objective is counted in independent experiments (e.g., at least three or seven independent experiments).

Other exemplary means for evaluating samples include a high throughput apparatus, such as the Amersham Biosciences IN Cell Analysis System and Cellomics™ ArrayScan HCS System which permit the subcellular location and concentration of fluorescently tagged moieties to be detected and quantified, both statically and kinetically. See also, U.S. Pat. No. 5,989,835.

Other exemplary mammalian cell lines include: a CHO cell line and a 293 cell line. For example, CHO cells with integrated copies of HD gene exon 1 with approximately 103Q repeats fused to GFP as a fusion construct encoding HD gene exon 1 Q103-GFP produce a visible GFP aggregation at the nuclear membrane, detectable by microscopy, whereas CHO cells with integrated copies of fusion constructs encoding HD gene exon 1 Q24-GFP in CHO cells do not produce a visible GFP aggregation at the nuclear membrane. In another example, 293 cells with integrated copies of the HD gene exon 1 containing 84 CAG repeats are used.

A number of animal model system for Huntington's disease are available. See, e.g., Brouillet, Functional Neurology 15(4): 239-251 (2000); Ona et al. Nature 399: 263-267 (1999), Bates et al. Hum Mol Genet. 6(10):1633-7 (1997); Hansson et al. J. of Neurochemistry 78: 694-703; and Rubinsztein, D. C., Trends in Genetics, Vol. 18, No. 4, pp. 202-209 (a review on various animal and non-human models of HD).

In one embodiment, the animal is a transgenic mouse that can express (in at least one cell) a human Huntingtin protein, a portion thereof, or fusion protein comprising human Huntingtin protein, or a portion thereof, with, for example, at least 36 glutamines (e.g., encoded by CAG repeats (alternatively, any number of the CAG repeats may be CAA) in the CAG repeat segment of exon 1 encoding the polyglutamine tract).

An example of such a transgenic mouse strain is the R6/2 line (Mangiarini et al. Cell 87: 493-506 (1996)). The R6/2 mice are transgenic Huntington's disease mice, which over-express exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N-terminal part of Huntingtin (encoded by HD exon 1), are observed in R6/2 mice, both in the cytoplasm and nuclei of cells (Davies et al. Cell 90: 537-548 (1997)). For example, the human Huntingtin protein in the transgenic animal is encoded by a gene that includes at least 55 CAG repeats and more preferably about 150 CAG repeats.

These transgenic animals can develop a Huntington's disease-like phenotype. These transgenic mice are characterized by reduced weight gain, reduced lifespan and motor impairment characterized by abnormal gait, resting tremor, hindlimb clasping and hyperactivity from 8 to 10 weeks after birth (for example the R6/2 strain; see Mangiarini et al. Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. Accordingly, evaluating can include assessing parameters related to neurotransmitter levels, neurotransmitter receptor levels, brain size and striatum size. In addition, abnormal aggregates containing the transgenic part of or full-length human Huntingtin protein are present in the brain tissue of these animals (e.g., the R6/2 transgenic mouse strain). See, e.g., Mangiarini et al. Cell 87: 493-506 (1996), Davies et al. Cell 90: 537-548 (1997), Brouillet, Functional Neurology 15(4): 239-251 (2000) and Cha et al. Proc. Natl. Acad. Sci. USA 95: 6480-6485 (1998).

To test the effect of the test compound or known compound described in the application in an animal model, different concentrations of test compound are administered to the transgenic animal, for example by injecting the test compound into circulation of the animal. In one embodiment, a Huntington's disease-like symptom is evaluated in the animal. For example, the progression of the Huntington's disease-like symptoms, e.g. as described above for the mouse model, is then monitored to determine whether treatment with the test compound results in reduction or delay of symptoms. In another embodiment, disaggregation of the Huntingtin protein aggregates in these animals is monitored. The animal can then be sacrificed and brain slices are obtained. The brain slices are then analyzed for the presence of aggregates containing the transgenic human Huntingtin protein, a portion thereof, or a fusion protein comprising human Huntingtin protein, or a portion thereof. This analysis can includes, for example, staining the slices of brain tissue with anti-Huntingtin antibody and adding a secondary antibody conjugated with FITC which recognizes the anti-Huntingtin's antibody (for example, the anti-Huntingtin antibody is mouse anti-human antibody and the secondary antibody is specific for human antibody) and visualizing the protein aggregates by fluorescent microscopy. Alternatively, the anti-Huntingtin antibody can be directly conjugated with FITC. The levels of Huntingtin's protein aggregates are then visualized by fluorescent microscopy.

A Drosophila melanogaster model system for Huntington's disease is also available. See, e.g., Steffan et al., Nature, 413: 739-743 (2001) and Marsh et al., Human Molecular Genetics 9: 13-25 (2000). For example, a transgenic Drosophila can be engineered to express human Huntingtin protein, a portion thereof (such as exon 1), or fusion protein comprising human Huntingtin protein, or a portion thereof, with, for example, a polyglutamine region that includes at least 36 glutamines (e.g., encoded by CAG repeats (preferably 51 repeats or more) (alternatively, any number of the CAG repeats may be CAA)) The polyglutamine region can be encoded by the CAG repeat segment of exon 1 encoding the poly Q tract. These transgenic flies can also engineered to express human Huntingtin protein, a portion thereof (such as exon 1), or fusion protein comprising human Huntingtin protein, or a portion thereof, in neurons, e.g., in the Drosophila eye.

The test compound (e.g., different concentrations of the test compound) or a compound described herein can be administered to the transgenic Drosophila, for example, by applying the pharmaceutical compositions that include the compound into to the animal or feeding the compound as part of food. Administration of the compound can occur at various stages of the Drosophila life cycle. The animal can be monitored to determine whether treatment with the compound results in reduction or delay of Huntington's disease-like symptoms, disaggregation of the Huntingtin protein aggregates, or reduced lethality and/or degeneration of photoreceptor neurons are monitored.

Neurodegeneration due to expression of human Huntingtin protein, a portion thereof (such as exon 1), or fusion protein comprising human Huntingtin protein, or a portion thereof, is readily observed in the fly compound eye, which is composed of a regular trapezoidal arrangement of seven visible rhabdomeres (subcellular light-gathering structures) produced by the photoreceptor neurons of each Drosophila ommatidium. Expression of human Huntingtin protein, a portion thereof (such as exon 1), or fusion protein comprising human Huntingtin protein, or a portion thereof, leads to a progressive loss of rhabdomeres. Thus, an animal to which a test compound is administered can be evaluated for neuronal degeneration.

Morely et al. (2002) Proc. Nat. Acad. USA Vol. 99:10417 describes a C. elegans system for evaluating Huntington's disease related protein aggregation.

Evaluting Huntington's Disease

A variety of methods are available to evaluate and/or monitor Huntington's disease. A variety of clinical symptoms and indicia for the disease are known. Huntington's disease causes a movement disorder, psychiatric difficulties and cognitive changes. The degree, age of onset, and manifestation of these symptoms can vary. The movement disorder can include quick, random, dance-like movements called chorea.

One method for evaluating Huntington's disease uses the Unified Huntington's disease Rating Scale (UNDRS). It is also possible to use individual tests alone or in combination to evaluate if at least one symptom of Huntington's disease is ameliorated. The UNDRS is described in Movement Disorders (vol. 11:136-142,1996) and Marder et al. Neurology (54:452-458, 2000). The UNDRS quantifies the severity of Huntington's Disease. It is divided into multiple subsections: motor, cognitive, behavioral, functional. In one embodiment, a single subsection is used to evaluate a subject. These scores can be calculated by summing the various questions of each section. Some sections (such as chorea and dystonia) can include grading each extremity, face, bucco-oral-ligual, and trunk separately.

Exemplary motor evaluations include: ocular pursuit, saccade initiation, saccade velocity, dysarthria, tongue protrusion, finger tap ability, pronate/supinate, a fist-hand-palm sequence, rigidity of arms, bradykinesia, maximal dystonia (trunk, upper and lower extremities), maximal chorea (e.g., trunk, face, upper and lower extremities), gait, tandem walking, and retropulsion. An exemplary treatment can cause a change in the Total Motor Score 4 (TMS-4), a subscale of the UHDRS, e.g., over a one-year period.

Certain Animal and Cell Models

In one aspect, the invention features an animal (e.g., a non-human animal, e.g., a non-human mammal, or an invertebrate, e.g., a nematode, or fly) that comprises a modification that alters lifespan regulation and a heterologous protein that includes at least 35 glutamines or a polyglutamine region. The modification that alters lifespan regulation can be environmental, genetic, or epigenetic.

For example, the heterologous protein can includes a polyglutamine repeat that includes at least 35 glutamines (e.g., at least 45, 50, 60, 70, or 80 glutamines). In one embodiment, the heterologous protein can also include all or part of a human protein that is a polyglutamine disease protein. For example, the heterologous protein includes at least 50 amino acid of the amino acid sequence of exon 1 of the human Huntingtin protein. Homologues of such human proteins can also be used. In another embodiment, the cell expresses an endogenous protein that includes a polyglutamine repeat that includes at least 35 glutamines. For example, the heterologous protein includes a fluorophore (e.g., the protein is a fluorescent protein, e.g., GFP, YFP, etc.) or other chromophoe. For example, the protein can be intrinsically fluorescent. An example of an environmental alteration is an alteration that increase or decreases the rate of aging accordingly to at least one age-associated parameter. For example, caloric restriction can be used to extend lifespan. Over-feeding and fat and obesity inducing treatments can be used to reduce lifespan. Other environmental alterations include stress, hydrogen peroxide, and so forth. Still other alterations include pharmaceutical interventions that increase or decrease a cellular activity, e.g., activity of a pathway or axis described herein.

An example of a genetic alteration is at least one substitution, insertion, or deletion in a gene, e.g., a genomic copy of a gene. A genetic alteration can also be created by a transgene, e.g., that can over express a transcript, produce an anti-sense transcript, or produce dsRNA. Some genetic alterations also knock-out, e.g., create a deletion or other inactivating mutation in a gene. The animal can include a genetic alteration that alters one or more genes (e.g., two, or three) genes that are involved in age-regulation or are otherwise age-associated. For example, the gene can be a component of a pathway or axis described herein (e.g., GH/IGF-1, AMPK, SIRT, Indy, and so forth).

An example of an epigenetic alteration is e.g., RNA interference (e.g., using dsRNA or siRNA).

As used herein, a “polyglutamine region” of a protein is a region of the protein that includes at least 15 consecutive glutamine residues, and is at least 90 or 95% glutamine. Typically, the region is 100% glutamine and includes at least 30, 35, 40, 506, 70, 80, or 90 residues. Regions with greater than 35 glutamines are more prone to aggregation. Absent other factors, the propensity for aggregation increases with repeat length.

In one aspect, the invention features a method of screening a compound (e.g., a small molecule, siRNA, drug, antibody, nucleic acid, gene therapy vector and so forth). The method includes providing a cell or animal that includes a protein with a polyglutamine region that is prone to aggregation (e.g., more than a corresponding wild-type protein). The cell or animal also is altered to have reduced or enhance lifespan regulation, e.g., by genetic, environmental, or epigenetic modification, e.g., as described herein, e.g., by altering an age-associated protein described herein.

The compound is contacted to the cell or animal and a property of the cell or animal is evaluated. For example, the evaluated property can relate to protein aggregation, a neurological (e.g., cognitive) property, or a property of one or more of the proteins or biochemical pathways described herein (e.g., GH/IGF-1, AMPK, SIRT, Indy, and so forth).

Screening systems in which the control (e.g., no test compound is contacted) has a reduced lifespan or reduce age-associated properties) can provide a useful sensitized system for detecting the ability of a test compound to affect a cell or organism (e.g., to affect polyglutamine aggregation). On the other hand, screening systems in which the control (e.g., no test compound is contacted) has a enhanced lifespan or increase age-associated properties) also can provide a useful system for detecting the ability of a test compound to affect a cell or organism (e.g., to affect polyglutamine aggregation), e.g., by detecting synergies between the test agent and the compound, which may not be apparent in a wild-type scenario.

In another aspect, the invention features a non-human organism that includes a deficiency in an age-associated protein and a heterologous nucleic acid encoding a protein with a polyglutamine repeat region that includes at least 35 glutamines. The organism can be an invertebrate organism (e.g., a Drosophila or nematode) or a vertebrate organism (e.g., a non-human mammalian organism such as a rodent, e.g., a transgenic rodent). In one embodiment, the deficiency is caused by a genetic mutation. In another embodiment, the deficiency is caused by RNAi. In one embodiment, the age-associated protein can be a SIRT protein, a protein that is directly regulated by a SIRT protein, or a protein that directly regulates a SIRT1 protein. Exemplary SIRT proteins include SIRT1, 2, 3, 4, 5, 6, and 7. In another embodiment, the age-associated protein can be a component of the AMPK axis (e.g.., as defined in 60/430,804). In another embodiment, the associated protein is a carboxylate transporter, e.g., an INDY transporter or an SLC13 family member.

In another aspect, the invention features cultured cell preparation that includes: an engineered mammalian cell that expresses a protein with a polyglutamine repeat region of at least 35 glutamines and includes a genetic alteration that enhances longevity or that sensitizes the cell (e.g., increases GH/IGF-1 axis activity, decreases SIRT1 activity, etc.). The cell preparation can also include a test compound or a modulator (e.g., an agonist or antagonist) of an age-associated protein. The preparation can be used in a method for evaluating a test compound or a library of test compound. The method can include contacting a test compound to cells in the preparation; and evaluating the cells for aggregation of the protein with the polyglutamine repeats or a symptom of protein aggregation or a symptom of aging.

The R6/2 mouse is a transgenic animal expressing exon 1 of the human Huntingtin gene with an expanded poly-CAG repeat (including, e.g., up to 120 glutamine residues). These mice show characteristic neurodegeneration, motor deficits and death at 12-15 weeks. Neurons in these animals also show the characteristic nuclear aggregates of poly-Q huntingtin protein. Thus, this represents a good model human Huntington's disease, as the human protein is the causative agent and both cell biology and behavioral consequences of the disease are conserved. The rapid decline of these animals allows for simple and quick readouts of the progression of the disease. These readouts include time to death, neurological behavioral deficits (mainly motor impairment), and histological measurements of nuclear aggregate formation. These animals can be used to screen compounds believed to act on the huntingtin protein or to effect it accumulation in neuronal nuclei.

Moreover, crossing R6/2 mice with other transgenic mice allows for rapid genetic screens to illuminate biochemical and genetic pathways influencing the progression of the disease. Crossing mutant mice (KOs, transgenics, knock-ins of catalytically dead mutant proteins) for genes in pathways that regulate or are otherwise associated with aging (e.g. SIRT family, growth hormone and its receptor, peptide regulators of growth hormone secretion, insulin and IGF proteins and their receptors plus peptide regulators of insulin and IGF secretion, SLC13 family, AMPK subunits, etc.). In one embodiment, progeny of such mice can be evaluated for the progress of an age-related disorder, e.g., Huntington's disease.

Observations of these exemplary models can be used for any polyglutamine-based disorder, e.g,. a neurodegenerative disorder. Exemplary neurodegenerative disorders include: Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12). In a particular embodiment, the neurodegenerative disorder is Huntington's disease. In another embodiment, it is a neurodegenerative disorder other than Huntington's disease or other than a disorder mediated by polyglutamine aggregation.

The organisms described herein may be deficient in the activity of any protein that is associated with aging, e.g., associated with the regulation of lifespan. For example, mutant or otherwise altered (e.g., RNAi treated or transgenic) organisms can include an alteration in a component that regulates lifespan or that directly interacts with such a component.

Other types of combinatorial systems include environmental treatment of an organism that is mutant or otherwise altered (e.g., RNAi treated or transgenic) with respect to an age-associated protein or an age-associated pathway (e.g., an AMPK or GH/IGF-1 axis pathway) or component thereof.

Exemplary environmental treatments include stress (e.g., oxidative stress, genotoxic stress, H₂O₂, heavy metal exposure), caloric restriction, and treatment with a drug, e.g., a histone deactylase inhibitor, or an inhibitor of the GH/IGF-1 axis.

### EXAMPLES

The invention is further described in the following examples, which do not limit the scope of the invention described in the claims.

In vitro Determination of GH/IGF-1 Antagonist Activity

A commonly used method to screen for compounds that affect GH secretion is to use the rat pituitary cell culture assay. In a typical experiment involving rat pituitary cell culture assays to determine a compound's effect on GH secretion, pituitary glands are aseptically removed from Wistar male rats (150-200 g) and cultures of pituitary cells are prepared according to Cheng et al. Endocrinology 124: 2791-2798, 1989. The cells are treated with various compounds and assayed for GH secreting activity as described by Cheng et al., infra.

Functional activity of the various compounds can be evaluated by measuring GH secretion from primary cultures of rat anterior pituitary cells (Yang et al. Proc. Natl. Acad. Sci. USA 95:10836-10841, 1998). Cells are isolated from rat pituitaries by enzymatic digestion with 0.2% collagenase and 0.2% hyaluronidase in Hanks' balanced salt solution. The cells are suspended in culture medium and adjusted to a concentration of 1.5 3 105 cells/ml and 1.0 ml of this suspension is placed in each well of a 24-well tray. Cells are maintained in a humidified 5% CO2/95% air atmosphere at 37° C. for 3-4 days. The culture medium consists of DMEM containing 0.37% NaHCO3, 10% horse serum, 2.5% fetal bovine serum, 1% nonessential amino acids, 1% glutamine, 1% nystatin, and 0.1 % gentamicin. Before testing compounds for their capacity to inhibit GH release, cells are washed twice 1.5 hr before and once more immediately before the start of the experiment with the above culture medium containing 25 mM Hepes (pH 7.4). Compounds are tested in quadruplicate by adding them in 1 ml of fresh medium to each well and incubating them at 37° C. for 2 hr followed by centrifugation at 2000×g for 15 min to remove any cellular material. The supernatant fluid is assayed for GH by a double antibody radioimmunoassay. For example, antibody to rat GH (anti-rat GH-RIA-5/AFP-411S, hormones for iodination (rat GH-I-6/AFP-5676B), and reference preparation (rat GH-RP-2/AFP-3190B) can be used for this assay.

Other means of determining effects of a compound on GH are to use cultured human fetal pituitary cells or cultured GH-adenoma cells collected from acromegalic patients. Isolation of cells and growth conditions may differ from that described above.

In another example, the superfused rat pituitary system can be used to evaluate antagonism of the GH/IGF-1 axis by a compound (Vigh and Schally, Peptides 5:241-247, 1984; Rekasi and Schally, Proc. Natl. Acad. Sci. USA 90:2146-2149, 1993). Briefly, anterior pituitary cells are dispersed as described above. The test compound is perfused through the rat pituitary cells for 9 minutes (3 mL) at various concentrations (10⁻⁷-10⁻⁹ M). After this 9 minute incubation, the cells are exposed to a mixture of the test compound and 10⁻⁹ M hGHRH¹⁻²⁹NH₂ for an additional 3 minutes. To check the duration of the antagonistic effect of the test compound, 10⁻⁹ M hGHRH¹⁻²⁹NH₂ is applied 30 and 60 minutes later for 3 minutes. GH content of the 1 mL fractions collected can be determined by double-antibody RIA (materials supplied by the National Hormone and Pituitary Program, Baltimore, Md.). Net integral values of the GH responses can be evaluated with a computer program designed for this use (Csernus, et al., Neuroendocrine Research Methods, ed. Greenstein (Harwood, London), 1991). GH responses can be compared to and expressed as a percentage of the original GH response induced by 10⁻⁹ M hGHRH¹⁻²⁹NH₂. The potencies of the test compounds can be compared to that of the standard GHRH antagonist (vide supra).

A number of embodiments of the invention have been described. Nevertheless, it will be understood that various modifications may be made without departing from the spirit and scope of the invention. Accordingly, other embodiments are within the scope of the following claims.

What is claimed:

1. A method of evaluating a compound for a modulatory effect on a disorder, the method comprising:
a) providing a library of compounds;
b) contacting each compound of the library to a GH/IGF-1 axis component or a functional fragment thereof, in vitro;
c) evaluating interaction between each compound and the GH/IGF-1 axis component;
d) selecting a subset of compounds from the library based on the evaluated interactions;
e) contacting a compound of the subset to (i) a cell in vitro, the cell being from a subject having the disorder or from non-human animal model of the disorder, or (ii) a non-human animal model of the disorder; and
f) evaluating the cell or the animal model, wherein a change in an parameter of the disorder identifies the respective compound as having a modulatory effect on the disorder.

2. The method of claim 1 wherein contacting the compound to the animal model comprises administering the compound to the animal model.

3. The method of claim 1 wherein the disorder is a neoplastic disorder, a neurological disorder, other than a disorder caused by polyglutamine aggregation, a metabolic disorder, an immunological disorder, a tissue repair condition, a dermatological disorder, a dermatological tissue condition, or a cardio-vascular disorder.

4. The method of claim 1 wherein the disorder is Alzheimer's, Parkinson's, ALS, skeletal muscle atrophy, multiple sclerosis, a neuropathy, age-related macular degeneration, diabetic retinopathy, or non-insulin-dependent diabetes.

5. The method of claim 1 wherein the component is a cell surface receptor or secreted molecule.

6. A method of evaluating a compound for a modulatory effect on a disorder, the method comprising:
a) selecting a GH/IGF-1 axis modulator;
b) contacting the modulator to (i) a cell in vitro, the cell being from a subject having the disorder or from non-human animal model of the disorder, or (ii) a non-human animal model of the disorder; and
c) evaluating the cell or the animal model, wherein a change in an parameter of the disorder identifies the respective compound as having a modulatory effect on the disorder, wherein the disorder is selected from the group consisting of: an immunological disorder, a dermatological disorder, a dermatological tissue condition, a cardiovascular disorder, or a neurological disorder, other than a neurological disorder caused by polyglutamine aggregation.

7. The method of claim 6 wherein the modulator is a compound that directly antagonizes a positively acting GH/IGF-1 axis component.

8. The method of claim 6 wherein the modulator is a compound that directly agonizes an inhibitory GH/IGF-1 axis component.

9. A method of evaluating a compound for a modulatory effect on life span regulation or potential, the method comprising
a) providing a test compound;
b) contacting the test compound to a GH/IGF-1 axis component in vitro;
c) evaluating interaction between the test compound and the GH/IGF-1 axis component;
d) administering the test compound to an adult, non-human subject; and
e) evaluating an age-associated parameter of the adult subject, wherein an interaction between the test compound the GH/IGF-1 axis component and modulation of the age-associated parameter relative to a control subject identifies the respective compound as having a modulatory effect on lifespan regulation or potential.

10. A method of evaluating a compound for a modulatory effect on life span regulation or potential, the method comprising
a) providing a library of compounds;
b) contacting each compound of the library to a GH/IGF-1 axis component in vitro;
c) evaluating interaction between each compound and the GH/IGF-1 axis component;
d) selecting a subset of compounds from the library based on the evaluated interactions;
e) administering (e.g., individually) each compound of the subset to an adult, non-human subject; and
f) evaluating an age-associated parameter of the adult subject, wherein modulation of the age-associated parameter relative to a control subject identifies the respective compound as having a modulatory effect on lifespan regulation or potential.

11. The method of claim 10, wherein the age-associated parameter comprises one or more of:
(i) lifespan of the subject, or a cell in the subject;
(ii) presence or abundance of a gene transcript or gene product that has a biological age-dependent expression pattern in a cell of the subject;
(iii) resistance of the subject or a cell of the subject to stress;
(iv) one or more metabolic parameters of the subject or a cell of the subject ; and
(v) proliferative capacity of a cell of the subject.

12. The method of claim 10, wherein the in vitro contacting is a cell-based assay.

13. The method of claim 10, wherein the in vitro contacting is a cell-free assay.

14. The method of claim 10, wherein the adult subject is a non-human mammal.

15. The method of claim 10, wherein the subject has normal IGF-1 levels.

16. The method of claim 10, the GH/IGF-1 axis component is a cell surface receptor.

17. The method of claim 10, the GH/IGF-1 axis component is a pre-IGF1 component.

18. The method of claim 10, the GH/IGF-1 axis component is a post-IGF1 component.

19. The method of claim 10 wherein the library comprises multiple compounds that have a molecular weight less than 7000 Daltons.

20. The method of claim 10 wherein the library comprises one or more of an immunoglobulin, a peptide, a nucleic acid aptamer, a dsRNA, a siRNA, a ribozyme, or an antisense nucleic acid.

21. The method of claim 10 wherein each compound of the library is non-polymeric.

22. The method of claim 10 further comprising formulating an identified compound as a pharmaceutical composition.

23. A method of evaluating a compound for a modulatory effect on life span regulation or potential, the method comprising
a) providing a test compound;
b) contacting the test compound to a GH/IGF-1 axis component in vitro;
c) evaluating interaction between the test compound and the growth hormone/IGF-1 axis component;
d) contacting the test compound to a cell; and
d) evaluating an age-associated parameter of the cell, wherein an interaction between the test compound the GH/IGF-1 axis component and modulation of the age-associated parameter relative to a control cell identifies the respective compound as having a modulatory effect on lifespan regulation or potential.

24. The method of claim 23, wherein the age-associated parameter comprises one or more of:
(i) lifespan of the cell;
(ii) presence or abundance of a gene transcript or gene product that has a biological age-dependent expression pattern in the cell;
(iii) resistance of the cell to stress;
(iv) one or more metabolic parameters of the cell
(v) proliferative capacity of the cell ; and
(vi) physical appearance or behavior of the cell.

25. A method identifying a GH/IGF-1 axis antagonist or partial agonist, the method comprising
a) providing a test compound that is obtained by chemically modifying an agonist of a GH/IGF-1 axis component or that is selected for structural similarity to an agonist of a GH/IGF-1 axis component; and
b) evaluating a property of a GH/IGF-1 axis component in vitro, in a cell, or in an organism in the presence of the test compound, wherein ability of the test compound to modulate the property of the GH/IGF-1 axis component identifies the test compound as a GH/IGF-1 axis antagonist.

26. The method of claim 25 wherein the evaluating comprises a cell-free assay or a cell-based assay.

27. The method of claim 25 wherein the evaluating comprises administering the test compound to an adult organism.

28. The method of claim 27 wherein the organism has normal IGF-1 levels prior to the administering.

29. The method of claim 27 wherein a cohort of adult organism are treated and evaluated, each organism of the cohort characterized by normal IGF-1 levels prior to the treating.

30. The method of claim 27 wherein the evaluating comprises evaluating GH or IGF-1 levels, and decreased levels of growth hormone and/or IGF-1 identifies the test compound as an agent as a modulator.

31. The method of claim 27 wherein the evaluating comprises evaluating activity of an GH/IGF-1 axis component in the organism.

32. The method of claim 25 further comprising d) evaluating an age-associated parameter of a subject treated with the test compound, wherein modulation the age-associated parameter relative to a control subject further identifies the test compound as an agent that modulates lifespan regulation or potential.

33. A method of identifying an agent that modulates lifespan regulation of an adult animal, the method comprising
a) selecting an agent that alters a property of GH/IGF-1 axis;
b) administering the agent to a subject; and
c) evaluating an age-associated parameter in the subject, wherein modulation of the age-associated parameter identifies the agent as an agent that modulates lifespan regulation or potential.

34. The method of claim 33 wherein the agent is a direct antagonist of a positively acting component of the GH/IGF-1 axis.

35. The method of claim 33 wherein the agent is a direct agonist of an inhibitor component of the GH/IGF-1 axis.
